(19) United States
(12) Patent Application Publication
(10) Pub. No.: US 2021/0213181 A1
(43) Pub. Date: Jul. 15, 2021

(54) Anti-Adhesive Shear Thinning Hydrogels

(71) Applicant: The Brigham and Women's Hospital, Inc., Boston, MA (US)

(72) Inventors: Ali Khademhosseini, Cambridge, MA (US); Guillermo Ulises Ruiz Esparza Herrera, Cambridge, MA (US); Xichi Wang, Brighton, MA (US); Su Ryon Shin, Brookline, MA (US); Thomas Jozefiak, Belmont, MA (US)

(21) Appl. No.: 17/058,224

(22) PCT Filed: May 29, 2019
(86) PCT No.: PCT/US2019/034298

§ 371 Date: Nov. 24, 2020

Related U.S. Application Data
(60) Provisional application No. 62/677,350, filed on May 29, 2018.

(51) Int. Cl. A61L 31/14; A61K 33/06; A61L 31/12

(52) U.S. Cl. CPC A61L 31/145; A61L 2400/06; A61L 31/128; A61K 33/06

(57) ABSTRACT

The present application relates to shear thinning hydrogel compositions which are useful for reducing and/or preventing tissue adhesion in a subject (e.g., a post-operative subject). Methods of using the compositions and kits comprising the compositions oar also provided.

### CROSS-REFERENCE TO RELATED APPLICATIONS

This application claims the benefit of U.S. Provisional Application Ser. No. 62/677,350, filed May 29, 2018, the disclosure of which is incorporated herein by reference in its entirety.

### FEDERALLY SPONSORED RESEARCH OR DEVELOPMENT

This invention was made with Government support under Grant Nos. 1R01EB023052, 1R01HL140618, 1R01HL137193, and 1R01GM126831, awarded by the National Institutes of Health. The Government has certain rights in the invention.

### TECHNICAL FIELD

The present application relates to shear thinning compositions which are useful for reducing and/or preventing tissue adhesion in a subject (e.g., a post-operative subject).

### BACKGROUND

More than 90% of surgical patients develop postoperative adhesions, and the incidence of hospital re-admission due to complications related to this pathology can be as high as 20%. Current adhesion barriers present limited efficacy and low clinical adoption due to difficulties in application on irregular surfaces, and incompatibility with minimally invasive interventions.

### SUMMARY

The present application provides, inter alia, a composition, comprising:

about 3% to about 15% by weight laponite; and

about 0.5% to about 10% by weight poly(ethylene oxide).

In some embodiments, the composition comprises about 3% to about 10% laponite. In some embodiments, the composition comprises about 5% to about 10% laponite. In some embodiments, the composition comprises about 8% to about 10% laponite. In some embodiments, the laponite comprises a positively charged edge and a negatively charged surface. In some embodiments, the overall charge of the laponite is negative.

In some embodiments, the laponite has an average diameter of from about 5 nm to about 60 nm. In some embodiments, the laponite has an average diameter of from about 10 nm to about 40 nm. In some embodiments, the laponite has an average diameter of from about 20 nm to about 30 nm.

In some embodiments, the laponite has an average thickness of from about 0.5 nm to about 2 nm. In some embodiments, the laponite has an average thickness of about 1 nm.

In some embodiments, the composition comprises about 1% to about 3% by weight poly(ethylene oxide). In some embodiments, the composition comprises about 3% to about 5% by weight poly(ethylene oxide). In some embodiments, the poly(ethylene oxide) has an average molecular weight of about 18,000 to about 22,000 g/mol.

In some embodiments, the composition comprises:

about 8% to about 10% by weight laponite and about 1% to about 5% by weight poly(ethylene oxide); or

about 5% to about 10% by weight laponite and about 1% to about 3% by weight poly(ethylene oxide); or

about 8% to about 10% by weight laponite and about 1% to about 3% by weight poly(ethylene oxide).

In some embodiments, the composition comprises:

about 5% by weight laponite and about 1% by weight poly(ethylene oxide); or

about 8% by weight laponite and about 1% by weight poly(ethylene oxide); or

about 10% by weight laponite and about 1% by weight poly(ethylene oxide); or

about 5% by weight laponite and about 2% by weight poly(ethylene oxide); or

about 8% by weight laponite and about 2% by weight poly(ethylene oxide); or

about 10% by weight laponite and about 2% by weight poly(ethylene oxide); or

about 5% by weight laponite and about 3% by weight poly(ethylene oxide); or

about 8% by weight laponite and about 3% by weight poly(ethylene oxide); or

about 10% by weight laponite and about 3% by weight poly(ethylene oxide).

In some embodiments, the composition further comprises water. In some embodiments, the composition comprises about 85% to about 94% by weight water.

In some embodiments, the composition comprises:

about 8% to about 10% by weight laponite, about 1% to about 5% by weight poly(ethylene oxide), and about 85% to about 91% by weight water; or

about 5% to about 10% by weight laponite, about 1% to about 3% by weight poly(ethylene oxide), and about 87% to about 94% by weight water; or

about 8% to about 10% by weight laponite, about 1% to about 3% by weight poly(ethylene oxide), and about 87% to about 91% by weight water.

In some embodiments, the composition consists of laponite, poly(ethylene oxide), and water.

In some embodiments, the water is deionized water.

In some embodiments, the composition is a gel. In some embodiments, the composition is a hydrogel.

In some embodiments, the laponite consists of about 66% SiO₂, about 30% MgO, about 3% Na₂O, and about 1% LiO₂.

The present application further provides a composition comprising about 5% to about 10% by weight laponite, about 1% to about 5% by weight poly(ethylene oxide), and about 85% to about 94% by weight water, wherein the composition is prepared according to a process comprising:

(a) combining the laponite and water to form a first mixture;

(b) adding the poly(ethylene oxide) to the first mixture to form the composition.

In some embodiments, the yield stress of the composition is from about 100 Pa to about 2000 Pa.

The present application further provides a kit comprising a composition provided herein. In some embodiments, the kit further comprises one or more sterile syringes. In some embodiments, the composition is preloaded into the one or more sterile syringes.

In some embodiments, the kit further comprises one or more sterile bandages. In some embodiments, the composition is preloaded onto a surface of the one or more sterile bandages.

In some embodiments, the kit further comprises one or more sterile surgical staples. In some embodiments, the composition is preloaded onto a surface of the one or more sterile surgical staples.

In some embodiments, the kit further comprises one or more sterile surgical sutures. In some embodiments, the composition is preloaded onto a surface of the one or more sterile surgical sutures.

The present application further comprises a sterile syringe comprising a composition provided herein.

The present application further comprises a sterile bandage, comprising a composition provided herein.

The present application further comprises a sterile surgical staple, comprising a composition provided herein.

The present application further comprises a sterile surgical suture, comprising a composition provided herein.

The present application further comprises a coating, comprising a pharmaceutically acceptable amount of a composition provided herein. In some embodiments, the coating is preloaded into a sterile syringe. In some embodiments, the coating is preloaded onto a surface of a sterile bandage. In some embodiments, the coating is preloaded onto a surface of a sterile surgical staple. In some embodiments, the coating is preloaded onto a surface of a sterile surgical suture.

The present application further provides a method of reducing or preventing tissue adhesion in a subject, comprising administering to the subject a composition provided herein.

In some embodiments, the tissue adhesion is associated with a surgical procedure, an injury, an anatomical defect, a cosmetic defect, or any combination thereof.

In some embodiments, the composition forms a barrier between two or more tissues in the subject, thereby reducing or preventing the tissue adhesion.

The present application further provides a method of reducing or preventing fibrotic adhesion formation in a subject, comprising administering to the subject a composition provided herein.

In some embodiments, the composition is administered by injection. In some embodiments, the composition is administered as a sprayable composition. In some embodiments, the composition is administered using a syringe.

In some embodiments, the method provided herein is associated with one or more of surgical, cosmetic, orthopedic, ophthalmic, and dermal applications.

In some embodiments, the composition provided herein is administered during a surgical procedure. In some embodiments, the surgical procedure is associated with one or more of abdominal, thoracic, pelvic, vascular, cardiovascular, neurological, and dermal surgical procedures.

In some embodiments, the method provided herein comprises local administration at a tissue associated with a surgical procedure, an injury, an anatomical defect, a cosmetic defect, or any combination thereof. In some embodiments, the surgical procedure is selected from the group consisting of a laparoscopic surgical procedure and an arthroscopic guided surgical procedure. In some embodiments, the surgical procedure is selected from the group consisting of laparoscopic myomectomy, ileal pouch anal-anastomosis, cesarean section, nerve repair, hernia repair, cardiac surgery, spinal surgery.

In some embodiments, the composition provided herein is administered using a catheter.

In some embodiments, the method comprises reducing or preventing the closure of permanent surgical central access lines, peripheral access lines, catheter lines, or drain lines, in the subject.

In some embodiments, the composition provided herein is locally administered at the site of a surgical suture or surgical staple in the subject.

In some embodiments, the composition forms a barrier between two or more tissues in the subject, thereby reducing or preventing the fibrotic adhesion formation.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### DESCRIPTION OF DRAWINGS

FIG. 1A shows a schematic representation of STHB formulation and delivery methods.

FIG. 1B shows the composition of STHB formulations using several silicate nanoplatelets (SNP)/poly(ethylene oxide) (PEO) ratios. Inherent viscosity was obtained by recording the maximum value during rheological shear rate sweeps after a 5-min equilibration at 37° C.

FIG. 1C shows viscosity versus shear rate. Viscosity decreased as shear rate increased illustrating the shear-thinning properties of the STHB compositions.

FIG. 1D shows strain (0.01 to 1000% at 1 Hz) versus storage moduli (G′), quantified to determine the linear viscoelastic regions (LVR) of STHB formulations. The plateau of G′ indicates the strain resistance of the compositions before deformation and transition to a liquid-like state.

FIG. 1E shows tan (δ) versus strain, calculated to determine the elastic to viscous transformation. The gel point (tan(δ)=1) was found at ˜10% strain.

FIG. 1F shows storage moduli (G′) recorded during multiple cycles of low (1%) and high (100%) strain. The light gray regions indicate rapid sample recovery to its original modulus.

FIG. 2A shows a schematic representation of the set-up used to characterize the injection force via a mechanical tester.

FIG. 2B shows injection force (N) as a function of time. Measurements were performed in newtons (N) and the plateau was used to determine the maximum required injection force to extrude the hydrogels.

FIG. 2C shows that Higher SNP concentration and smaller needle intraluminal diameters resulted in higher required injection force (n=5) FIG. 2D shows a schematic representation of the system used to spray STHB.

FIG. 2E shows STHB spraying performed from a 22 G nozzle at a 1 mL/min flow rate and 100 kPa.

FIG. 2F shows total spray area measured after 0.1 mL of STHB was applied. Formulations with increased SNP concentration exhibited smaller spray areas. Data is represented in means SD, n=3. P-values were determined by Student t-test (ns: P>0.05).

FIG. 2G shows spot distribution of STHB formulations. Scale bar=2 cm.

FIG. 2H shows average spot size. Higher viscosity formulations resulted on increased average spot areas. Data is represented in means SD, n=3. P-values were determined by Student t-test (ns: P>0.05).

FIG. 2I shows schematic representation of the setup used to determine and measure the spreadability of STHB compositions.

FIG. 2J shows STHB formulations with higher SNP concentration resulted in less spread area. The inclusion of PEO in the compositions did not have any effect in the spreadability. Data is represented in means SD, n=3. P-values were determined by Student t-test (ns: P>0.05).

FIG. 2K shows that STHB formulations remained stable with minimal degradation after 21 days. Data is represented in means±SD, n=3. P-values were determined by Student t-test (ns: P>0.05).

FIG. 2L shows the Swelling ratios of STHB formulations, determined over a course of 21 days. Maximum swelling occurred after 3 days of incubation. Data is represented in means±SD, n=3. P-values were determined by Student t-test (ns: P>0.05).

FIG. 3A shows a schematic representation of the test used to determine adherence and morphological features of fibroblasts seeded in the surface of STHB formulations.

FIG. 3B shows cell viability determined using relative fluorescence units to correlate cell numbers. The inclusion of 3 wt % PEO on STHB formulations decreased the cell adherence to hydrogel surfaces significantly. Data is represented in means SD, n=5. P values determined by one-way analysis of variance (ANOVA).

FIG. 3C shows representative fluorescent micrographs of cellular morphological features after 24 hours of incubation on the surface of PTFE substrates (control), SNP STHB formulations (5L, 8L, 10L), and SNP/PEO STHB formulations (5L3P, 8L3P, 10L3P). In the presence of SNP/PEO STHB compositions, fibroblasts (red) exhibited limited pseudopodia expansion. Scale bar=20 μm. Data is represented in means SD, n=5. P values determined by one-way analysis of variance (ANOVA).

FIG. 3D shows cellular morphology analyzed by quantifying the cell area and aspect ratio of the fibroblasts seeded on PTFE substrates (control), SNP STHB formulations (5L, 8L, 10L), and SNP/PEO STHB formulations (5L3P, 8L3P, 10L3P). SNP/PEO STHB compositions presented a reduced cell area and aspect ratio (gray area) (n>60)

FIGS. 3E-3G show cytotoxicity evaluated in fibroblasts after 48 hours of incubation on increasing SNP, PEO and SNP/PEO concentrations (0.001 to 1000 μg/mL). Decreased cell viability was observed when SNP concentration was above 100 μg/mL (FIG. 3E). No cytotoxicity was observed when cells were exposed to PEO (FIG. 3F). Viability was not affected when cells were exposed to a combination of SNPs and PEO (FIG. 3G). Data is represented in means SD, n=5. P values determined by one-way analysis of variance (ANOVA).

FIG. 4A shows a schematic representation of a rat peritoneal adhesion model. Eight ischemic buttons were created by grasping and ligating parietal peritoneum and STHB was applied to coat the button.

FIG. 4B shows the peritoneal adhesion index (PAI) used to grade postoperative adhesion formation.

FIG. 4C shows representative images of the application of Seprafilm® and STHB formulations (5L3P, 8L3P and 10L3P) on the peritoneal ischemic buttons. A sham group was used as a control. STHB formulations were easily delivered at the injury site achieving the formation of a resistant barrier. Black arrows indicate ischemic buttons and delivery site.

FIG. 4D shows a reopened incision (after 14 days) evaluated for adhesion formation. STHB formulations resulted in decreased adhesion formation compared to Seprafilm® and control. Black arrows indicate postoperative adhesions.

FIG. 4E shows average adhesion score calculated using the PAI scoring system. 10L3P group presented the lower score. Data is represented in means SD, n=5. P values determined by one-way analysis of variance (ANOVA).

FIG. 4F shows the average adhesion formation (%) per rat calculated by percentage of buttons affected. STHB formulations resulted in less amount of adhesion formation. Data is represented in means SD, n=5. P values determined by one-way analysis of variance (ANOVA).

FIG. 4G shows normalized average efficacy, defined as the decrease (%) of adhesion formation compared to the control. STHB formulations presented superior performance compared to Seprafilm®. Data is represented in means SD, n=5. P values determined by one-way analysis of variance (ANOVA).

FIG. 5A shows representative micrographs of hematoxylin and eosin (H&E) staining from postoperative adhesion tissue (insets represent magnified tissue areas). The interface between the injured peritoneal lining and fibrotic tissue can be observed in control, Seprafilm®, 5L3P, and 8L3P groups. On the 10L3P group, the peritoneal lining was preserved, and no adhesions were detected. Black arrows indicate adhesion formation areas. Scale bars on micrographs are 1 mm on the left picture, and 200 μm on the magnified insets in the right.

FIG. 5B shows results of Masson's trichrome staining performed in all groups. Muscular tissue can be observed in red and collagen in blue (white asterisks); highly organized fibrotic collagen bands were identified on control, Seprafilm® and 5L3P. The more viscous STHB formulations (8L3P and 10L3P) presented a decreased and more homogenous collagen distribution as observed on the micrographs and their respective magnified insets. Black arrows indicate adhesion formation areas. Scale bars on micrographs are 1 mm on the left picture, and 200 μm on the magnified insets in the right.

FIG. 5C shows results of immunohistochemistry performed in all the groups to determine macrophage (CD68, green) and lymphocyte (CD3, red) infiltration in response to the materials. Nuclear staining (DAPI) is shown in blue. Minimal localized immune infiltration was found in all the groups indicating negligible host immune response against the materials. Scale bars on the fluorescent micrographs are 500 μm.

FIGS. 6A-6F shows viscosity versus shear rate characterized on STHB compositions with 0, 1, and 2 wt % PEO. Rheological analysis showed that formulations with higher SNP concentrations resulted in higher viscosities and stronger gel formation (FIGS. 6A-6F). The addition of 1 and 2 wt % PEO to the compositions did not impact the viscosity or shear-thinning properties, however, a slightly decrease was observed when 3 wt % PEO was introduced. In all cases, no significant effect on their shear-thinning behavior was observed (FIGS. 6D-6F).

FIGS. 7A-7C show results of linear viscoelastic region determination on STHB compositions with 0, 1, and 2 wt % PEO. Strain (0.01 to 1000% at 1 Hz) versus storage modulus (G′) was quantified and the linear viscoelastic region (LVR) identified as the G′ plateau (0.1-10 strain (%)). LVR was similar in all the compositions.

FIGS. 8A-8C show gel point of STHB compositions with 0, 1, and 2 wt % PEO. Tan (δ) versus strain was calculated to determine the elastic to viscous transformation, and gel point defined as tan(δ)=1. The gel point of all formulations was found consistent at ˜10% strain.

FIGS. 9A-9C show rapid mechanical recovery after applied strain on STHB compositions with 0, 1, and 2 wt % PEO. Storage moduli (G′) was recorded during multiple cycles at low (1%) and high (100%) strain. All formulations presented rapid recovery to its original modulus (light gray regions) after strain was removed.

FIGS. 10A-10C show injection force of STHB formulations measured by extruding the hydrogels through 18 G (FIG. 10A), 23 G (FIG. 10B), and 27 G (FIG. 10C) needles at an infusion rate of 2 mL/min. Higher concentration of SNPs and smaller needle intraluminal diameter resulted in higher injection force; the addition of PEO did not have any significant influence on the injection force. Data is represented in mean±SD (n=5). The required injection force to extrude the hydrogel using 18 G needles varied from 2.09 to 3.28 N for formulations that contained 5 wt % SNPs (5L, 5L1P, 5L2P, and 5L3P), 4.59 to 6.74 N for formulations that contained 8 wt % SNPs (8L, 8L1P, 8L2P, and 8L3P), and 7.87 to 8.81 N for formulations that contained 10 wt % SNPs (10L, 10L1P, 10L2P, and 10L3P) as shown in FIG. 10A. For 23 G needles the required extrusion force was 5.13 to 6.61 N for formulations that contained 5 wt % SNPs (5L, 5L1P, 5L2P, and 5L3P), 11.42 to 13.23 N for formulations that contained 8 wt % SNPs (8L, 8L1P, 8L2P, and 8L3P), and 17.39 to 19.18 N for formulations that contained 10 wt % SNPs (10L, 10L1P, 10L2P, and 10L3P) as shown in FIG. 10B. Finally, 27 G needles required an extrusion force in the ranges of 9.75 to 14.00 N for formulations that contained 5 wt % SNPs (5L, 5L1P, 5L2P, and 5L3P), 19.36 to 21.49 N for formulations that contained 8 wt % SNPs (8L, 8L1P, 8L2P, and 8L3P), and 30.94 to 31.69 N for formulations that contained 10 wt % SNPs (10L, 10L1P, 10L2P, and 10L3P) as shown in FIG. 10C.

FIGS. 11A-11B show results of a cell adherence assessment. To determine the number of attached cells, a quantification of relative fluorescence units emitted by cells seeded on the surface of STHB formulations with 0 wt % (5L, 8L, 10L), 1 wt % (5L1P, 8L1P, 10L1P) and 2 wt % (5L2P, 8L2P, 10L2P) PEO was performed, a PTFE substrate was used as a control. The presence of PEO in STHB formulations decreased cell adherence. Data is represented in mean±SD (n=5). P-values were determined by Student t-test (ns: P>0.05). As compared to 5L, 8L, 10L (SNP-only formulations), a decrease in cell numbers of 19.3%, 23.2%, and 16.7% was present in 5L1P, 8L1P, 10L1P (1 wt % PEO compositions) as shown in FIG. 11A. In the case of 2 wt % PEO formulations, a similar result was obtained, as 5L2P presented a decrease of 16.6% in cell attachment, 8L2P a decrease of 17.8%, and 10L2P a decrease of 20.4%, compared to 5L, 8L, and 10L, respectively, as shown in FIG. 11B.

FIGS. 12A-12B show adhesion grade distribution on ischemic buttons. The total amount of adhesions by grade was quantified according to the PAI scoring system (FIG. 12A). Average number of adhesions per grade per animal were calculated; adhesion severity was decreased with Seprafilm® and STHB formulations. 10L3P was the formulation that showed superior performance with no adhesion formation. Data is represented in mean SD (FIG. 12B). During the procedure, STHB application was easier than Seprafilm®, as fragility and limited adaption of the Seprafilm® to irregular surfaces increased the application time compared to the facile delivery of STHB formulations. Data shows that control group had a similar distribution of adhesions for each grade with an average of 2.6 ischemic buttons with grade 1 adhesions, 2.0 with grade 2, 1.0 with grade 3, and 2.4 with no adhesions. The formation of postoperative adhesions secondary to Seprafilm® application were related to low-grade adhesions with an average of 3.2 ischemic buttons for grade 1, 0.8 for grade 2, 0.0 for grade 3 and 4.0 without any adhesion. 5L3P presented a similar distribution of adhesions of grade 2 and 3, averaging 0.6 for grade 1, 1.2 for grade 2, 1.2 for grade 3, and 5.0 with no adhesions, and 8L3P mainly presented grade 1 and 3 adhesions, with an average of 0.6 for grade 1, 0.0 for grade 2, 0.6 for grade 3 and 6.8 without any adhesion; 10L3P did not present any adhesions (averaging 8 ischemic buttons with grade 0). Based on these results it was concluded that Seprafilm®, 5L3P and 8L3P had similar adhesion formation levels (by grade), and 10L3P was the formulation that showed superior performance compared to all the groups as calculated by the PAI scoring system.

### DETAILED DESCRIPTION

A challenging medical problem associated with surgical intervention has been the formation of adhesions, as about 93% of patients who undergo open pelvic or abdominal surgery develop this pathology (see e.g., Menzies & Ellis, Ann. R. Coll. Surg. Engl. 1990, 72(1); 60-63; Parker et al, Colorectal. Dis. 2004, 6(6):506-511; and Parker et al, Dis. Colon Rectum 2001, 44(6):822-829). Postoperative adhesions are pathologic formations of fibrotic tissue that occur after peritoneal injury, and adhere the inner peritoneal lining of the abdominopelvic wall to internal organs within the abdominal or pelvic cavities (intestines, liver, gallbladder, urinary bladder, uterus, fallopian tubes, ovaries) (see e.g., Arung et al, World J. Gastroenterol. 2011, 17(41):4545-4553). A multifactorial cascade that involves ischemia, inflammation, angiogenesis, and tissue repair is known to cause its formation (see e.g., Reed et al, J. Surg. Res. 2002, 108(1):165-172; and Holmdahl et al, Eur. J. Surg. 1999, 165(11):1012-1019). Adhesions are associated with significant decreased quality of life, morbidity and mortality, and the incidence of hospital re-admissions due to its complications (intestinal obstruction, chronic abdominopelvic pain and secondary infertility) is as high as 20% (see e.g., Menzies & Ellis, Ann. R. Coll. Surg. Engl. 1990, 72(1); 60-63; Parker et al, Colorectal. Dis. 2004, 6(6):506-511; and Parker et al, Dis. Colon Rectum 2001, 44(6):822-829; Ouaissi et al J. Visc. Surg. 2012, 149(2), e104-114; Klingensmith et al, Surg. Endosc. 1996, 10(11):1085-7; Hallfeldt et al, Zentralbl. Chir. 1995, 120(5):387-391; Kolmorgen et al, Zentralbl. Gyakol. 1991, 113(6): 291-295; Swank et al, Lancet 2003, 361(9365):1247-1251; Tulandi et al, Am. J. Obstet. Gynecol. 1990, 162(2):354-357; Marana et al, Hum. Reprod. 1999, 14(12):2991-2995; Milingos et al, Ann. N.Y. Acad. Sci. 2000, 900:272-285; and Vrijland et al, Surg. Endosc. 2003, 17(7):1017-1022.

Physical barriers in the form of films have been reported for use in preventing adhesions, however, their application to irregular surfaces and cavities is challenging or impossible, as the films are fragile, difficult to handle, incompatible with minimally invasive laparoscopic or catheter-based procedures, and limited efficacy (e.g., ˜25%) decreases clinical adoption (see e.g., Wilson, M. S. Colorectal. Dis. 2007, 9 Suppl. 2:60-65; and Hirschelmann et al, Arch. Gynecol. Obster. 2012, 285(4):1089-1097). The development of new technologies to solve these clinical limitations is desirable.

Hydrogel formulations for the prevention of postoperative adhesions may be particularly attractive as a substitute for commercially available barriers. Without being bound by theory, an exemplary hydrogen biomaterial would include mechanical and biological properties to prevent cell adherence, infiltration, and adhesion formation. The hydrogel would also be injectable to be compatible with minimally invasive procedures such as laparoscopies and arthroscopies, and sprayable to uniformly cover large and irregular areas during laparotomies or thoracotomies, a limitation that current ‘film-based’ barriers have.

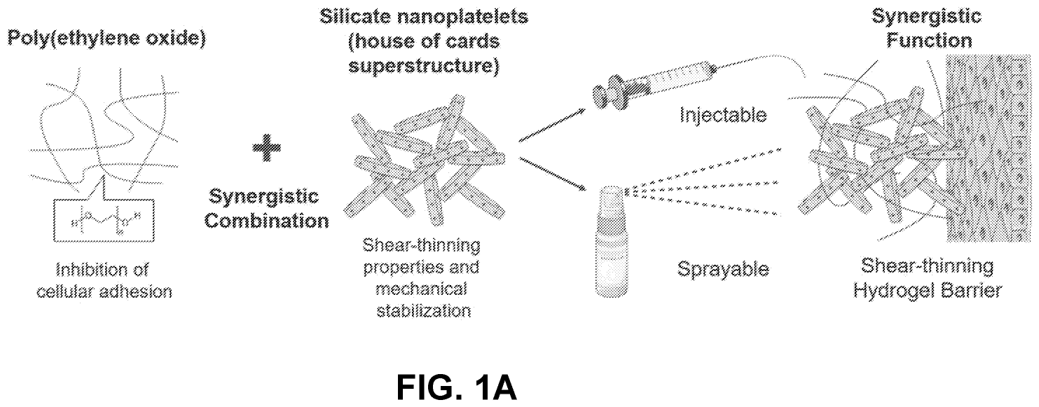

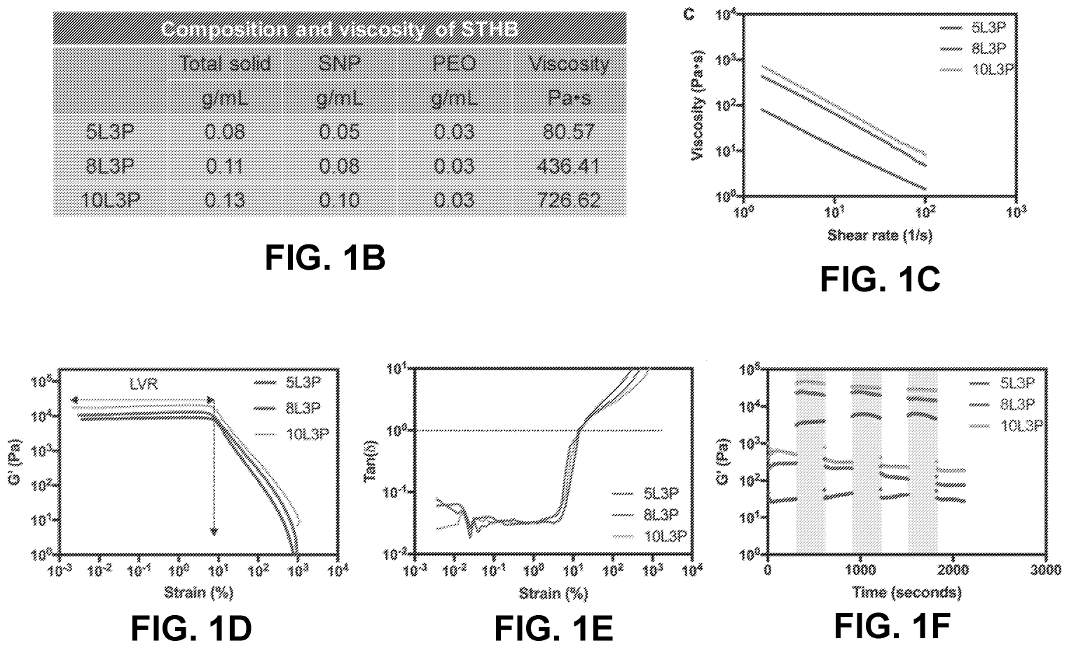

To achieve the desired mechanical and biological properties, the present application provides a hydrogel composed of silicate nanoplatelets (SNP) and poly(ethylene oxide) (PEO). The disc-shaped SNPs (e.g., thickness ˜0.92 nm and diameter ˜25 nm) possess unique electrostatic properties with negatively charged surfaces and positively charged edges that result in a nanoscale surface-to-edge attraction and spontaneous formation of a superstructure. The dual electrostatic charges in the surface of SNPs nanoplatelets confer non-Newtonian and shear-thinning behavior to the material, allowing its injectability and sprayability after subjecting the material to stress, with subsequent mechanical recovery immediately after delivery (FIG. 1A) (see e.g., Gaharwar et al, ACS. Nano. 2014, 8(10):9833-9842). Additionally, the self-assembly properties of SNPs allow for formation of a nanostructured ultra-efficient barrier, providing an organized tortuous network that prevents and reduces the transport of molecules and colloidal particles (see e.g., Yoo et al, Nanoscale, 2014, 6(18):10824-10830). PEO, a biocompatible and Food and Drug Administration (FDA) approved polymer for drug delivery systems was selected for combination with the SNPs, as the PEO exhibits antifouling properties with minimal binding sites for cell adherence or protein adsorption, thereby prevent cellular infiltration and growth (see e.g., Mazunin et al, ACS Biomaterials Science & Engineering 2015, 1(6):456-462; and Alcantar et al, J Biomed. Mater. Res. 2000, 51(3):343-351).

Without being bound by theory, it was hypothesized that the synergistic combination of the mechanical and biological features of SNPs and PEO would provide a shear-thinning hydrogel barrier (STHB) that can be administered via multiple facile delivery methods (e.g., spraying, injecting, spreading, and the like). The compositions provided herein were designed to provide physical separation between tissues and inhibit infiltration of collagen-secreting cells that lead to adhesion formation, thereby providing a universal solution to prevent the formation of postoperative adhesions over a wide range of surgical procedures.

### Compositions

Accordingly, the present application provides compositions comprising silicate nanoplatelets (SNPs) and poly(ethylene oxide). In some embodiments, the present application provides compositions comprising laponite (an exemplary SNP) and poly(ethylene oxide). In some embodiments, the compositions provided herein exhibit shear-thinning behavior (i.e., the compositions are shear-thinning compositions). The expression “shear-thinning” or “shear-thinning behavior”, refers to a decrease in viscosity (i.e., increasing flow rate) of a composition with increasing application of shear stress. For example, a shear-thinning composition (i.e. a composition exhibiting shear-thinning behavior) can exhibit a decrease in viscosity (i.e. increase in flow) upon application of an increasing rate of shear stress.

In some embodiments, the composition comprises about 3% to about 12% by weight laponite, for example, about 3% to about 10%, about 3% to about 8%, about 3% to about 5%, about 5% to about 12%, about 5% to about 10%, about 5% to about 8%, about 8% to about 12%, about 8% to about 10%, or about 10% to about 12% by weight laponite. In some embodiments, the composition comprises about 3% to about 10% laponite. In some embodiments, the composition comprises about 5% to about 10% laponite. In some embodiments, the composition comprises about 8% to about 10% laponite.

In some embodiments, the laponite comprises a positively charged edge and a negatively charged surface. In some embodiments, the overall charge of the laponite is negative.

In some embodiments, the laponite has an average diameter of from about 5 nm to about 60 nm, for example, about 5 to about 40, about 5 to about 20, about 5 to about 10, about 10 to about 60, about 10 to about 40, about 10 to about 20, about 20 to about 60, about 20 to about 40, or about 40 to about 60 nm. In some embodiments, the laponite has an average diameter of from about 10 nm to about 40 nm. In some embodiments, the laponite has an average diameter of from about 20 nm to about 30 nm.

In some embodiments, the laponite has an average thickness of from about 0.5 nm to about 2 nm, for example, about 0.5 to about 1.5, about 0.5 to about 1, about 0.5 to about 0.75, about 0.75 to about 2, about 0.75 to about 1.5, about 0.75 to about 1, about 1 to about 2, about 1 to about 1.5, or about 1.5 to about 2 nm. In some embodiments, the laponite has an average thickness of about 1 nm.

In some embodiments, the laponite provided herein comprises about 66% SiO₂, about 30% MgO, about 3% Na₂O, and about 1% LiO₂. In some embodiments, the laponite provided herein consists of about 66% SiO₂, about 30% MgO, about 3% Na₂O, and about 1% LiO₂.

In some embodiments, the composition comprises about 0.5% to about 10% by weight poly(ethylene oxide) (PEO), for example, about 0.5% to about 8%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 8%, about 1% to about 6%, about 1% to about 5%, about 1% to about 4%, about 1% to about 3%, about 1% to about 2%, about 2% to about 10%, about 2% to about 8%, about 2% to about 6%, about 2% to about 5%, about 2% to about 4%, about 2% to about 3%, about 3% to about 10%, about 3% to about 8%, about 3% to about 6%, about 3% to about 5%, about 3% to about 4%, about 4% to about 10%, about 4% to about 8%, about 4% to about 6%, about 4% to about 5%, about 6% to about 10%, about 6% to about 8%, or about 8% to about 10%, by weight poly(ethylene oxide). In some embodiments, the composition comprises about 1% to about 5% by weight poly(ethylene oxide). In some embodiments, the composition comprises about 1% to about 3% by weight poly(ethylene oxide). In some embodiments, the composition comprises about 3% to about 5% by weight poly(ethylene oxide).

In some embodiments, the poly(ethylene oxide) has an average molecular weight of about 18,000 to about 22,000 g/mol, for example, about 18,000 to about 21,000, about 18,000 to about 20,000, about 18,000 to about 19,000, about 19,000 to about 22,000, about 19,000 to about 21,000, about 19,000 to about 20,000, about 20,000 to about 22,000, about 20,000 to about 21,000, about 21,000 to about 22,000 g/mol.

In some embodiments, the composition comprises:

about 3% to about 10% by weight laponite; and

about 1% to about 10% by weight poly(ethylene oxide).

In some embodiments, the composition comprises:

about 5% to about 10% by weight laponite; and

about 1% to about 5% by weight poly(ethylene oxide).

In some embodiments, the composition comprises:

about 8% to about 10% by weight laponite and about 1% to about 5% by weight poly(ethylene oxide); or

about 5% to about 10% by weight laponite and about 1% to about 3% by weight poly(ethylene oxide); or

about 8% to about 10% by weight laponite and about 1% to about 3% by weight poly(ethylene oxide).

In some embodiments, the composition comprises:

about 5% by weight laponite and about 1% by weight poly(ethylene oxide); or

about 8% by weight laponite and about 1% by weight poly(ethylene oxide); or

about 10% by weight laponite and about 1% by weight poly(ethylene oxide); or

about 5% by weight laponite and about 2% by weight poly(ethylene oxide); or

about 8% by weight laponite and about 2% by weight poly(ethylene oxide); or

about 10% by weight laponite and about 2% by weight poly(ethylene oxide); or

about 5% by weight laponite and about 3% by weight poly(ethylene oxide); or

about 8% by weight laponite and about 3% by weight poly(ethylene oxide); or

about 10% by weight laponite and about 3% by weight poly(ethylene oxide).

In some embodiments, the composition comprises:

about 5% by weight laponite and about 1% by weight poly(ethylene oxide); or

about 8% by weight laponite and about 2% by weight poly(ethylene oxide); or

about 10% by weight laponite and about 3% by weight poly(ethylene oxide).

In some embodiments, the composition comprises about 10% by weight laponite and about 3% by weight poly(ethylene oxide).

In some embodiments, the compositions provided herein further comprise water.

In some embodiments, the composition comprises about 80% to about 95% by weight water, for example, about 80% to about 90%, about 80% to about 85%, about 85% to about 95%, about 85% to about 90%, or about 90% to about 95% by weight water. In some embodiments, the composition comprises about 85% to about 94% by weight water. In some embodiments, the water is deionized water. In some embodiments, the water is ultra-pure water (e.g., Milli-Q) or buffered water (e.g., phosphate buffered saline).

In some embodiments, the composition comprises:

about 8% to about 10% by weight laponite, about 1% to about 5% by weight poly(ethylene oxide), and about 85% to about 91% by weight water; or

about 5% to about 10% by weight laponite, about 1% to about 3% by weight poly(ethylene oxide), and about 87% to about 94% by weight water; or

about 8% to about 10% by weight laponite, about 1% to about 3% by weight poly(ethylene oxide), and about 87% to about 91% by weight water.

In some embodiments, the composition provided herein consists of laponite, poly(ethylene oxide), and water.

In some embodiments, the composition provided herein is a gel. In some embodiments, the composition provided herein is a hydrogel. As used herein, the term “hydrogel” refers to a gel in which the liquid component comprises water.

The present application further provides a process of preparing a composition provided herein, the process comprising:

(a) combining laponite and water to form a first mixture; and

(b) adding the poly(ethylene oxide) to the first mixture to form the composition.

In some embodiments, the composition provided herein is prepared according to a process described herein. For example, in some embodiments, the present application provides a compositions comprising about 5% to about 10% by weight laponite, about 1% to about 5% by weight poly(ethylene oxide), and about 85% to about 94% by weight water, wherein the composition is prepared according to a process comprising:

(a) combining the laponite and water to form a first mixture;

(b) adding the poly(ethylene oxide) to the first mixture to form the composition.

In some embodiments, the yield stress of the composition provided herein is from about 100 Pa to about 2000 Pa, for example, about 100 Pa to about 1500 Pa, about 100 Pa to about 1000 Pa, about 100 Pa to about 750 Pa, about 100 Pa to about 500 Pa, about 100 Pa to about 250 Pa, about 100 Pa to about 200 Pa, about 200 Pa to about 2000 Pa, about 200 Pa to about 1500 Pa, about 200 Pa to about 1000 Pa, about 200 Pa to about 750 Pa, about 200 Pa to about 500 Pa, about 200 Pa to about 250 Pa, about 250 Pa to about 2000 Pa, about 250 Pa to about 1500 Pa, about 250 Pa to about 1000 Pa, about 250 Pa to about 750 Pa, about 250 Pa to about 500 Pa, about 500 Pa to about 2000 Pa, about 500 Pa to about 1500 Pa, about 500 Pa to about 1000 Pa, about 500 Pa to about 750 Pa, about 750 Pa to about 2000 Pa, about 750 Pa to about 1500 Pa, about 750 Pa to about 1000 Pa, about 1000 Pa to about 2000 Pa, about 1000 Pa to about 1500 Pa, or about 1500 Pa to about 2000 Pa.

In some embodiments, a shear-thinning composition provided herein flows upon application of a pressure greater than the yield stress, for example, application of a pressure about 10% greater, about 20% greater, about 30% greater, about 40% greater, about 50% greater about 60% greater, about 70% greater, about 80% greater, about 90% greater, or about 100% greater than the yield stress.

In some embodiments, a composition provided herein is self-healing. As used herein, the expression “self-healing”, refers to recovery of the elastic gel strength of a composition upon removal of a stress. In some aspects, a self-healing composition may recover elastic gel strength from about 2 seconds to about 1 minute after removal of a stress, for example, from 30 seconds to 1 min., from 30 seconds to 45 seconds, from 15 seconds to 1 minute, from 15 seconds to 45 seconds, from 15 seconds to 30 seconds, from 10 seconds to 15 seconds, from 10 seconds to 30 seconds, from 10 seconds to 45 seconds, from 10 seconds to 1 minute, from 5 seconds to 10 seconds, from 5 seconds to 25 seconds, from 5 seconds to 45 seconds, from 5 seconds to 1 minute, from 2 seconds to 10 seconds, from 2 seconds to 25 seconds, from 2 seconds to 45 seconds, or from about 2 seconds to 1 minute.

As used herein, the term “consisting essentially of” is used to define a composition or method that includes materials, steps, features, components, or elements, in addition to those explicitly provided herein, provided that these additional included materials, steps, features, components, or elements do not materially affect the basic and novel characteristic(s) of the claimed invention. In some embodiments, the additional materials, steps, feature, components, or elements do not affect the mode of action to achieve the desired result of the invention described herein. The term “consists essentially of” or “consisting essentially of” occupies a middle ground between “comprising” and “consisting of”.

In some embodiments, the present application provides a composition consisting essentially of silicate nanoplatelets (SNPs), poly(ethylene oxide), and one or more additional components. In some embodiments, the present application provides a composition consisting essentially of laponite, poly(ethylene oxide), and one or more additional components. In some embodiments, the present application provides a composition consisting essentially of silicate nanoplatelets (SNPs), poly(ethylene oxide), water, and one or more additional components (e.g., one or more excipients, buffering agents, additional therapeutic agents, and the like). In some embodiments, the one or more additional components do not materially affect the shear-thinning characteristics of the compositions described herein. In some embodiments, the one or more additional components do not materially affect the anti-adhesion characteristics of the compositions described herein. In some embodiments, the one or more additional components do not materially affect the shear-thinning and anti-adhesion characteristics of the compositions described herein.

For the terms “for example” and “such as” and grammatical equivalences thereof, the phrase “and without limitation” is understood to follow unless explicitly stated otherwise. As used herein, the term “about” is meant to account for variations due to experimental error. All measurements reported herein are understood to be modified by the term “about”, whether or not the term is explicitly used, unless explicitly stated otherwise. As used herein, the singular forms “a,” “an,” and “the” include plural referents unless the context clearly dictates otherwise.

The following abbreviations may be used throughout the present application: STHB: Shear-Thinning Hydrogel Barrier; SNP: Silicate Nanoplatelets (e.g., laponite); PEO: Poly(ethylene oxide); FDA: Food and Drug Administration; LVR: Linear Viscoelastic Region; PTFE: Polytetrafluoroethylene; PA: Peritoneal Adhesion Index; H&E: Hematoxylin and eosin.

### Methods of Use

The present application further provides methods of reducing (e.g., reducing the likelihood) and/or preventing tissue adhesion in a subject. In some embodiments, the method comprises administering the composition to the subject (e.g., an amount effective to reduce and/or prevent tissue adhesion in the subject). In some embodiments, the method is a method of reducing tissue adhesion in the subject. In some embodiments, the method is a method is preventing tissue adhesion in the subject.

As used herein, the term “subject,” refers to any animal, including mammals. For example, mice, rats, other rodents, guinea pigs, rabbits, dogs, cats, swine, cattle, sheep, horses, primates, and humans. In some embodiments, the subject is a human.

In some embodiments, the tissue adhesion is associated with a surgical procedure, an injury, an anatomical defect, a cosmetic defect, or any combination thereof. In some embodiments, the tissue adhesion is associated with a surgical procedure. In some embodiments, the tissue adhesion is associated with an injury. In some embodiments, the tissue adhesion is associated with an anatomical defect. In some embodiments, the tissue adhesion is associated with a cosmetic defect.

In some embodiments, the composition provided herein, upon administration to the subject, forms a barrier between two or more tissues in the subject, thereby reducing or preventing the tissue adhesion.

The present application further provides a method of reducing or preventing fibrotic adhesion formation in a subject. In some embodiments, the method comprises administering the composition to the subject (e.g., an amount effective to reduce and/or prevent fibrotic adhesion formation in the subject). In some embodiments, the method is a method of reducing fibrotic adhesion formation in the subject. In some embodiments, the method is a method is preventing fibrotic adhesion formation in the subject. In some embodiments, the composition forms a barrier between two or more tissues in the subject, thereby reducing or preventing the fibrotic adhesion formation.

In some embodiments, the method provided herein is associated with one or more of surgical, cosmetic (e.g., a cosmetic surgical procedure), orthopedic, ophthalmic, and dermal applications. In some embodiments, the composition is administered during a surgical procedure.

In some embodiments, the surgical procedure is associated with one or more procedures selected from abdominal, thoracic, pelvic, vascular, cardiovascular, neurological, and dermal surgical procedures. In some embodiments, the surgical procedure is selected from the group consisting of a laparoscopic surgical procedure and an arthroscopic guided surgical procedure. In some embodiments, the surgical procedure is selected from the group consisting of laparoscopic myomectomy, ileal pouch anal-anastomosis, cesarean section, nerve repair, hernia repair, cardiac surgery, spinal surgery.

In some embodiments, the method comprises local administration at a tissue associated with a surgical procedure, an injury, an anatomical defect, a cosmetic defect, or any combination thereof. In some embodiments, the method comprises local administration at a tissue associated with a surgical procedure. In some embodiments, the method comprises local administration at a tissue associated with an injury. In some embodiments, the method comprises local administration at a tissue associated with an anatomical defect. In some embodiments, the method comprises local administration at a tissue associated with a cosmetic defect.

As used here, the term “local administration” refers to administration at or within close proximity to the site at which the tissue adhesion is to be reduced and/or prevented in the subject. For example, upon cessation of the administration, a composition provided herein will remain substantially localized at the site of the administration. In some embodiments, the composition is locally administered at the site of a surgical suture or surgical staple in the subject.

In some embodiments, the composition is administered onto one or more surfaces of the subject (e.g., one or more tissues, one or more organs, and the like). In some embodiments, the composition is administered onto one or more internal surfaces of the subject (e.g., one or more tissues, one or more organs, and the like). In some embodiments, the composition is administered onto the skin of the subject.

In some embodiments, the method comprises reducing or preventing the closure of permanent surgical central access lines, peripheral access lines, catheter lines, or drain lines, or any combination thereof, in the subject.

The amount of the composition administered to a subject will vary depending upon the composition being administered, the purpose of the administration, the state of the subject, the manner of administration, and the like. Effective doses will depend on the necessary treatment as well as by the judgment of the attending clinician depending upon factors such as the type and severity of the complication, the age, weight and general condition of the subject, and the like.

### Cosmetic Applications

The compositions provided herein may also be useful as components in cosmetic compositions. Benefits of shear-thinning compositions in cosmetic applications include, but are not limited to, the biocompatibility and non-toxic character of the compositions provided herein (e.g., a shear-thinning composition may degrade over time in subcutaneous tissue). Exemplary cosmetic applications include, but are not limited to, cosmetic surgical procedures such as dental augmentation, breast augmentation, buttock augmentation, lip augmentation, jaw augmentation, hip augmentation, chin augmentation, brow augmentation, arm augmentation, leg augmentation, and the like. In some embodiments, the composition provided herein is administered during a cosmetic surgical procedure.

### Administration Routes

The compositions provided herein can be administered by a variety of routes, depending upon the area to be treated. In some embodiments, the composition is an injectable composition and/or a sprayable composition. In some embodiments, the composition is an injectable composition. In some embodiments, the composition is a sprayable composition.

In some embodiments, the composition is administered by parenteral administration. Parenteral administration includes intravenous, intraarterial, subcutaneous, intraperitoneal intramuscular, injection, or infusion; or intracranial, (e.g., intrathecal or intraventricular administration).

In some embodiments, the composition is administered by injection. In some embodiments, the composition is administered using a syringe. In some embodiments, the composition is administered using a catheter.

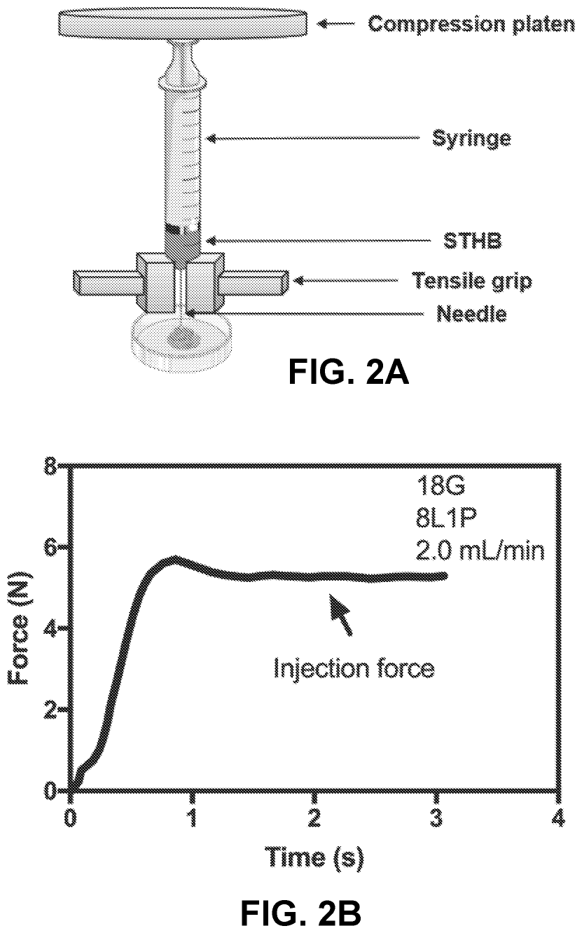

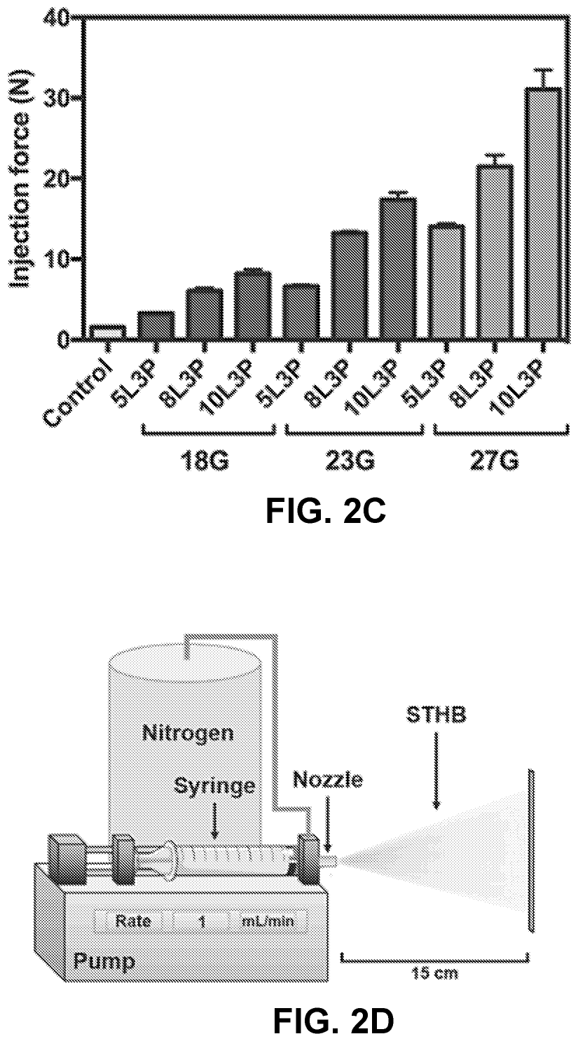

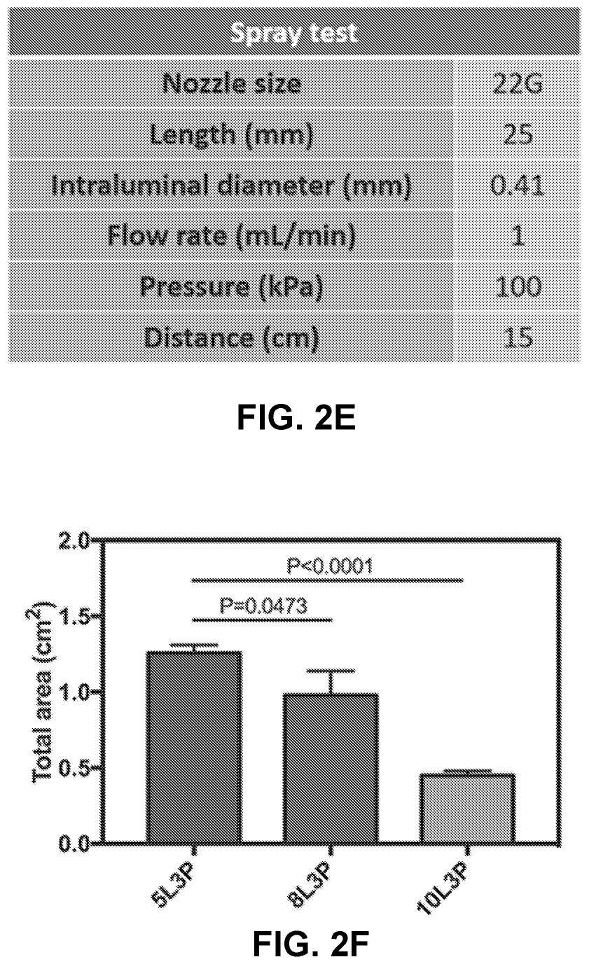

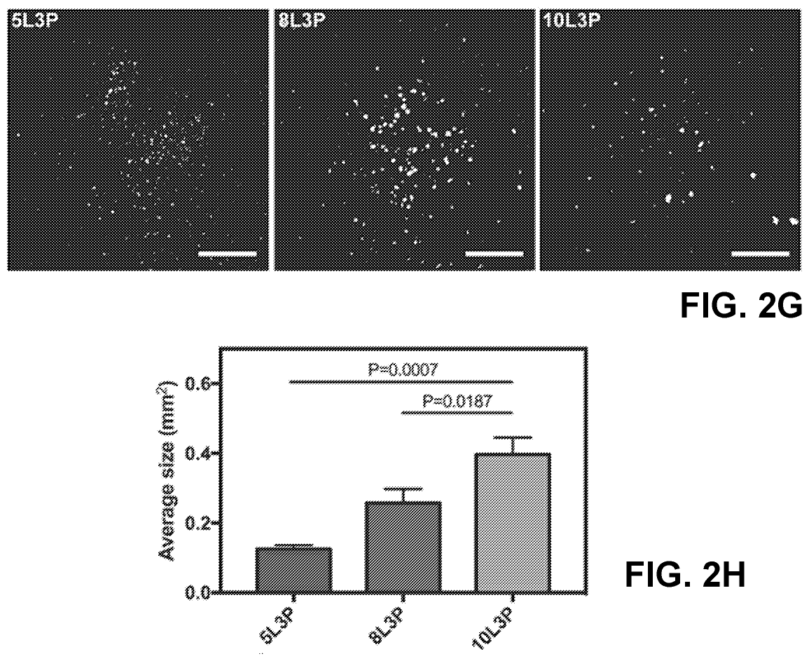

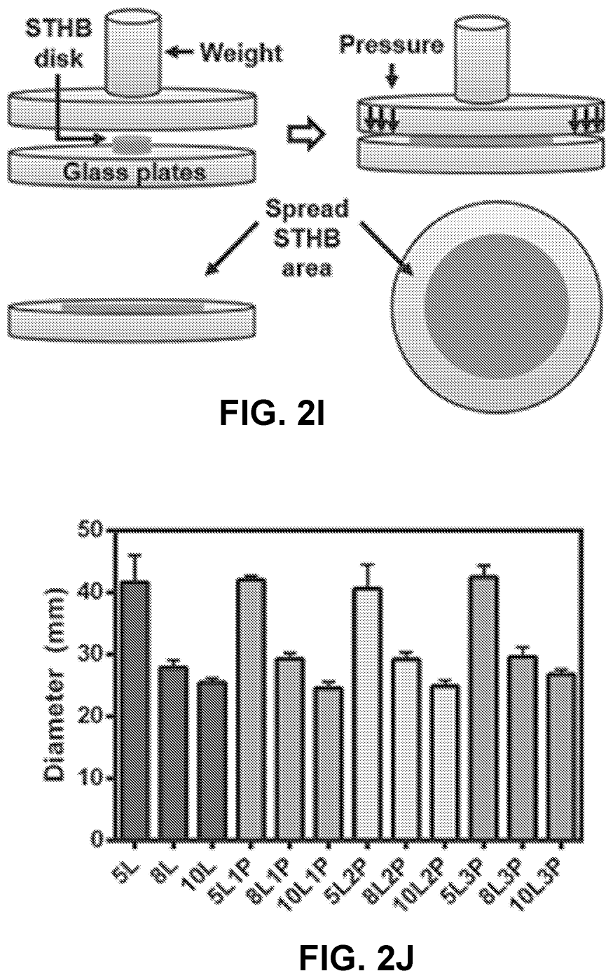

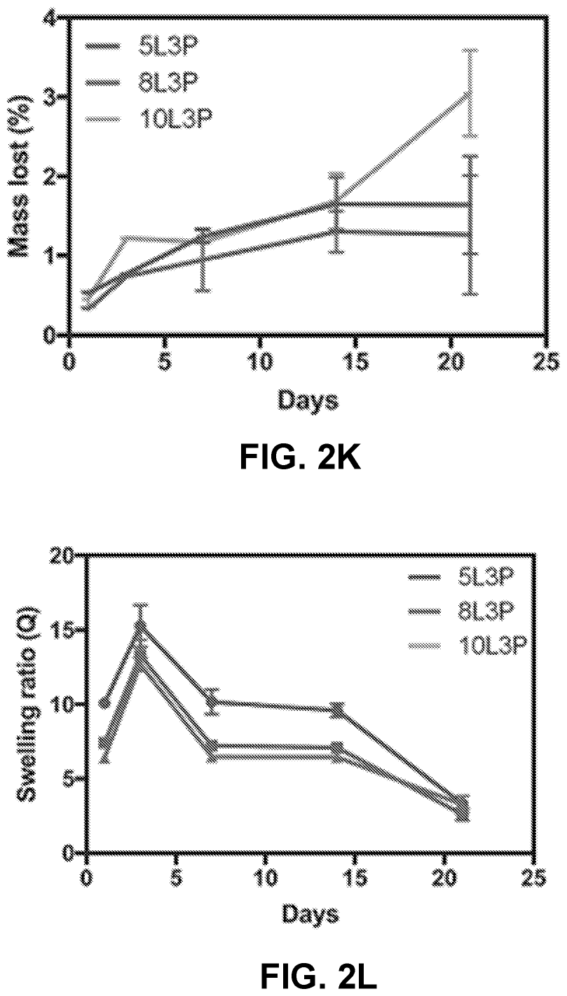

In some embodiments, the composition is preloaded onto at least one surface of a medical device (e.g., a syringe, catheter, surgical stable, medical wipe, bandage, a device suitable for spraying the composition (e.g., a container substantially similar to the container shown in FIG. 2D, or a compressed gas container) and the like) prior to administering to the subject. In some embodiments, the composition is preloaded into a medical device prior to administration to the subject. In some embodiments, the composition is preloaded into a medical device suitable for spraying the composition (e.g., a compressed gas container in combination with a compressed gas useful as a blowing agent for spraying the composition), prior to administration to the subject. In some embodiments, the shear-thinning composition is preloaded into a catheter prior to administering to the subject. In some embodiments, the shear-thinning composition is preloaded into a syringe prior to administering to the subject.

The present application further provides a medical device comprising a compound provided herein. Exemplary medical derives include, but are not limited to syringes, staples, bandages, catheters, and the like. The present application further provides a syringe (e.g., a sterile syringe) comprising a composition provided herein. The present application further provides a bandage (e.g., a sterile bandage) comprising a composition provided herein. The present application further provides a staple (e.g., a sterile staple or a sterile surgical staple) comprising a composition provided herein. In some embodiments, the staple is first applied to the subject, and the composition is subsequently administered to one or more surfaces of the staple. The present application further provides a suture (e.g., a sterile suture or a sterile surgical suture) comprising a composition provided herein. In some embodiments, the suture is first applied to the subject, and the composition is subsequently administered to one or more surfaces of the suture.

In some embodiments, the compositions provided herein are administered in the form of a coating (e.g., a spreadable coating). In some embodiments, the coating is preloaded into a syringe (e.g., a sterile syringe). In some embodiments, the coating is preloaded onto a surface of a bandage (e.g., a sterile bandage). In some embodiments, the coating is preloaded onto a surface of a staple (e.g., a sterile staple or a sterile surgical staple). In some embodiments, the coating is preloaded onto a surface of a suture (e.g., a sterile suture or a sterile surgical suture).

### Kits

The present application further provides a kit comprising a composition provided herein. In some embodiments, the kit further comprises one or more medical devices described herein. In some embodiments, the components of the kit can be separately packaged or contained.

In some embodiments, the kit further comprises one or more syringes (e.g., one or more sterile syringes). In some embodiments, the composition is preloaded into the one or more syringes. In some embodiments, the composition is preloaded into the one or more devices suitable for spraying the composition.

In some embodiments, the kit further comprises one or more bandages (e.g., one or more sterile bandages). In some embodiments, the composition is preloaded onto a surface of the one or more bandages.

In some embodiments, the kit further comprises one or more staples (e.g., one or more sterile staples). In some embodiments, the composition is preloaded onto a surface of the one or more staples.

In some embodiments, the kit further comprises one or more sutures (e.g., one or more sterile sutures or one or more sterile surgical sutures). In some embodiments, the composition is preloaded onto a surface of the one or more sutures.

In some embodiments, the kit further comprises one or more catheters (e.g., one or more sterile catheters). In some embodiments, the composition is preloaded into the one or more catheters.

Instructions, either as inserts or as labels, indicating quantities of the composition to be administered, guidelines for administration, guidelines for mixing components of the composition, and/or guidelines for preparing a composition according to a process described herein, can also be included in a kit provided herein. In some embodiments, the instructions further comprise instructions for performing one or more of the methods provided herein. In some embodiments, the instructions further comprise instructions for quantities of the composition to be administered, guidelines for administration, guidelines for mixing components of the composition, and/or guidelines for preparing a composition according to a process described herein.

The kits provided herein can further include, if desired, one or more conventional pharmaceutical kit components, such as, for example, containers with one or more pharmaceutically acceptable carriers, additional containers, etc., as will be readily apparent to those skilled in the art.

### EXAMPLES

The invention will be described in greater detail by way of specific examples. The following examples are offered for illustrative purposes, and are not intended to limit the invention in any manner.

### Cell Line and Cell Cultures

3T3 fibroblasts (ATCC, Manassas, Va.) were cultured in Dulbecco's modified eagle medium (DMEM) (Invitrogen, Carlsbad, Calif.) supplemented with 10% fetal bovine serum (FBS) (Atlanta Biologicals, Flowery Branch, Ga.) and 1% penicillin-streptomycin (PS) (MediaTech Inc., Manassas, Va.) under 5% CO₂ at 37° C. Cells were passaged approximately 2 times per week and media was exchanged every 2 days.

### Statistical Analysis

All the results are expressed as mean±SD. An unpaired Student t test was used to determine statistical significance of all samples and groups. *P<0.05, **P<0.01 will be considered statistically significant.

### Example 1. STHB Formulation

Biomaterials with shear-thinning properties are essential for the development of viscoelastic gel coatings that transform their mechanical properties to liquid-like states upon delivery under shear stress. This property enables their injectability or sprayability as a flowable liquid, and subsequent recovery to their original viscoelastic solid state when shear is removed after delivery. Previous attempts to provide sprayable hydrogel coatings as adhesion barriers have employed in-situ chemistry (polymerization or crosslinking) to achieve the desired viscoelastic properties of hydrogel coatings delivered from a liquid precursor formulation. The STHB properties described herein enable the development of injectable and sprayable hydrogel compositions for standard and minimally invasive medical interventions without the need of additional polymerization or crosslinking (see e.g, Gaharwar et al, ACS Nano. 2014, 8(10):9833-9842; and Avery et al, Sci. Transl. Med. 2016, 8(365):365ra156). STHB technology achieves these goals with far greater simplicity and elegance than 2-part in-situ crosslinked chemical systems.

STHBs were formulated with different percentages of poly(ethylene oxide) (PEO) (Sigma-Aldrich, St. Louis, Mo.) and silicate nanoplatelets (SNPs) Laponite XLG (BYK, Wesel, Germany). PEO and SNPs were sterilized using UV light and dissolved in ultra-filtered deionized water in separate vials. For dissolution, SNPs were stirred (400 rpm) at 60° C., and after 1 minute of stirring, PEO solution was added and stirred for four additional minutes until complete homogenization was achieved. The hydrogel compositions that were fabricated are 5 wt % SNP (5L), 8 wt % SNP (8L), 10 wt % SNP (10L), 5 wt % SNP 1 wt % PEO (5L1P), 8 wt % SNP 1 wt % PEO (8L1P), 10 wt % SNP 1 wt % PEO (10L1P), 5 wt % SNP 2 wt/o PEO (5L2P), 8 wt % SNP 2 wt % PEO (8L2P), 10 wt % SNP 2 wt % PEO (10L2P), 5 wt % SNP 3 wt % PEO (5L3P), 8 wt % SNP 3 wt % PEO (8L3P), and 10 wt % SNP 3 wt % PEO (10L3P) (see e.g., (FIG. 1B and Table 1). Clear hydrogel solutions were obtained and stored at room temperature for 48 hours to achieve stabilization.

TABLE 1

| Composition | Total Solid | SNP | PEO | Viscosity |
|---|---|---|---|---|
| ID | (g/mL) | (g/mL) | (g/mL) | Pa · s |
| 5L | 0.05 | 0.05 | — | 91.54 |
| 8L | 0.08 | 0.08 | — | 532.63 |
| 10L | 0.10 | 0.10 | — | 1010 |
| 5L1P | 0.06 | 0.05 | 0.01 | 131.55 |
| 8L1P | 0.09 | 0.08 | 0.01 | 536.91 |
| 10L1P | 0.11 | 0.10 | 0.01 | 1084 |
| 5L2P | 0.07 | 0.05 | 0.02 | 122.33 |
| 8L2P | 0.10 | 0.08 | 0.02 | 426.12 |
| 10L2P | 0.12 | 0.10 | 0.02 | 1037 |
| 5L3P | 0.08 | 0.05 | 0.03 | 80.57 |
| 8L3P | 0.11 | 0.08 | 0.03 | 436.41 |
| 10L3P | 0.13 | 0.10 | 0.03 | 726.62 |

### Example 2. Rheological Characterization of STHB Formulations

After formulation, the rheological properties of all STHB compositions were determined, and higher viscosity and stronger gel formation was observed on formulations with higher SNP concentrations (see e.g., FIG. 1B and Table 1) (see e.g, Zebrowski et al, Colloids and Surfaces A: Physicochemical and Engineering Aspects 2003, 213(2):189-197).

To characterize the rheology and mechanical properties of STHB formulations, a MCR 301 rheometer was used (Anton Paar, Graz, Austria) as previously described (see e.g., Gaharwar et al, ACS Nano. 2014, 8(10):9833-9842). Temperature sweeps were performed on a 25-mm diameter plate (gap height: 500 μm), and mineral oil was placed around to prevent water evaporation. Equilibration time was set to 10 min before testing, followed by steady shear at 10 s⁻¹ for 2 minutes. Shear rate sweeps (0.001 to 100 s⁻¹ with 10 points/decade) and strain sweeps (0.01 to 1000% at 1 Hz) were performed at 37° C. Recovery testing was performed by applying a value outside of the linear viscoelastic range (100% strain), followed by a value inside of the linear viscoelastic range (1% strain) at 1 Hz.

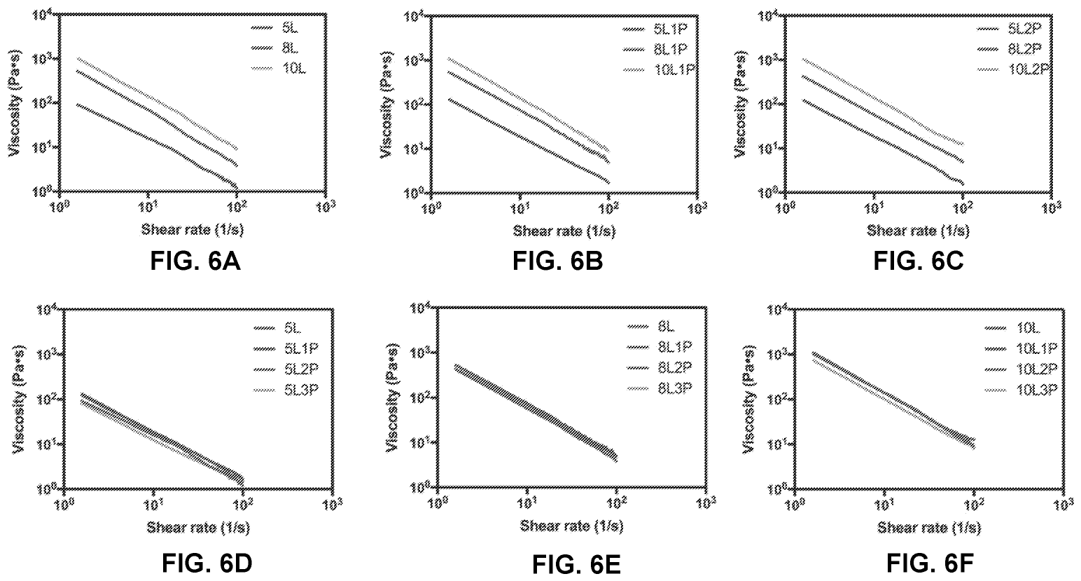

It was found that 10L3P presented higher viscosity (˜727 Pa·s) compared to formulations with lower SNP concentration, where 5L3P and 8L3P displayed a viscosity of ˜81 Pa·s and ˜436 Pa·s when subjected to a shear of ˜1.6 s⁻¹, as shown in FIG. 1C. A shear-thinning behavior was observed in all formulations as their viscosity decreased when shear rate was increased as shown in FIGS. 6A-6F (see e.g., Schmidt et al, Macromolecules, 2000, 33(20):7219-7222; Pozzo & Walker, Colloids and Surfaces A: Physicochemical and Engineering Aspects 2004, 240(1):187-198; and Fall & Bonn, Soft Matter 2012, 8(17):4645-4651).

### Example 3. Injection Force Test

The injection force required to extrude STHB formulations was analyzed using a mechanical tester (Instron Model 5542) (Instron, Norwood, Mass.). STHB formulations were loaded into 3-mL syringes (BD Biosciences, San Jose, Calif.) and injected through 3 different intraluminal diameter needle sizes, 0.838 mm (18 G), 0.337 mm (23 G), and 0.210 mm (27 G) (BD Biosciences, San Jose, Calif.). The syringe plungers were pressed by an upper compressive platen, and the lower housing of the syringe was placed into the tensile grip of the instrument to prevent movement. The injection rate used was 2 mL/min and the force on the plunger was measured with a 100-N load cell. All STHB samples were tested in triplicate. Bluehill version 3 software (Instron, Norwood, Mass.) was used to analyze the data.

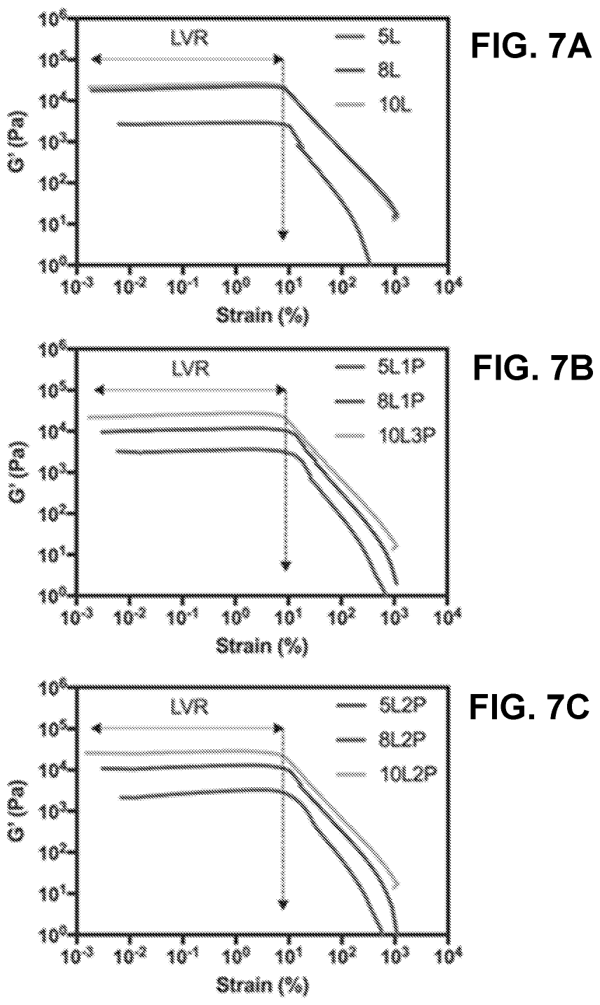

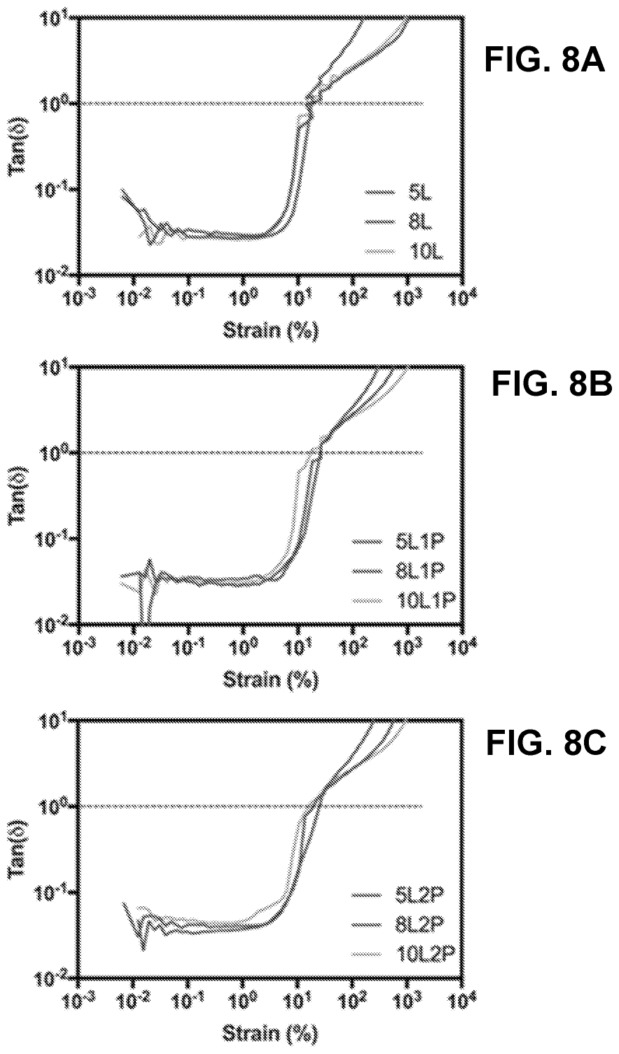

The viscoelastic behavior of STHBs was further characterized by strain sweeps (0.01 to 1000% at 1 Hz). The linear viscoelastic region (LVR) of STHBs was obtained within a small strain region (0.1-10.0 strain (%) at 1 Hz), as shown in FIG. 1D and FIGS. 7A-7C. Higher SNP content resulted in an increase of elastic modulus (G′) [e.g. 5L3P and 10L3P, had an average of ˜8002 and ˜17470 Pa at ˜0.1 strain (%) at 1 Hz]. Posterior to the LVR (˜10>strain (%) at 1 Hz), a fast decrease of G′ was observed from the disruption of the physical crosslinking of the gel through nanoplatelets disassembly (see e.g, Zuidema et al, J. Biomed. Mater. Res. B Appl. Biomater. 2014, 102(5), 1063-1073). A similar behavior was observed in the tan (δ) vs strain test, where the tan (δ) represents G″/G′; the gel point [tan (δ)=1] was detected at ˜10 strain (%), and higher elasticity was observed during a strain (%) of <10 (tan (δ)<1), with higher viscosities appearing during a strain (%) of >10 (tan (δ)>1), as shown in FIG. 1E and FIGS. 8A-8C), and as reported in previous studies (see e.g., Gaharwar et al, Macromol. Biosci. 2012, 12(6):779-93).

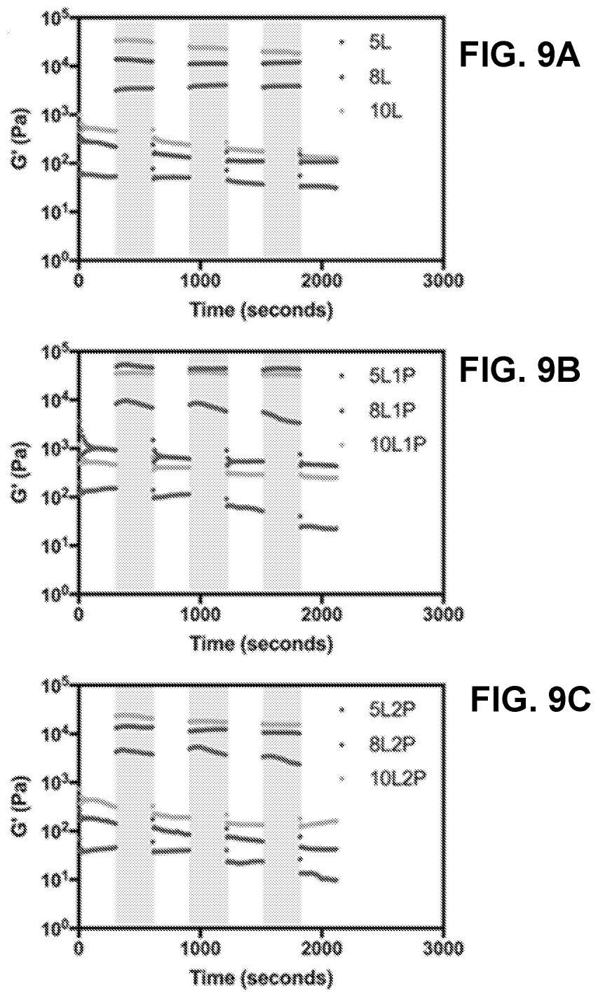

Low (1%) and high (100%) strain at 1 Hz was applied to STHBs over multiple cycles, demonstrating self-recovery to their original modulus, as shown in FIG. 1F and FIGS. 9A-C). The shear-thinning behavior of STHBs is caused by the ability of SNPs to disassemble and reassemble to its original structure when shear stress is applied and removed (see e.g., Labanda et al, Colloids and Surfaces A: Physicochemical and Engineering Aspects 2007, 301(1):8-15). The addition of PEO (1-3 wt %) to SNPs did not affect their shear-thinning behavior, suggesting the conservation of electrostatic interactions into the nanoplatelets surface.

### Example 4. Sprayability of STHB Formulations

To determine the sprayability of STHB formulations and facilitate visualization, hydrogel compositions were labeled with Alexa Fluor 488 dye (Thermo Fisher Scientific, Waltham, Mass.). STHB formulations were loaded into 10 mL syringes (BD, Franklin Lakes, N.J.) and sprayed through a specialized syringe extrusion setup was mounted on a mechanical testing instrument as shown in FIG. 2A). The setup consisted on an infusion pump (Harvard Apparatus, Holliston, Mass.) to control the hydrogel extrusion rate, and a STHB-loaded syringe with dual nozzles, one connected to a pressurized nitrogen tank and a second one used to spray the hydrogel compositions. The parameters used to spray STHB formulations were as follow, the pressure used to infuse the hydrogels was 100 kPa, the infusion rate was 1 mL/min, the spraying distance was set to 15 cm, and a 22 G nozzle with 0.41 mm of intraluminal diameter was used. Photographs and videos were taken during the spraying of 0.1 mL of the compositions, afterwards, the spray area and spot size were quantified by performing area fraction analysis using ImageJ software (National Institutes of Health, Bethesda, Md.) as previously reported (see e.g., Sakuma et al, Chemistry Letters 2018, 47(1):68-70).

The injectability of STHBs was evaluated on three needles with different intraluminal diameters (18 G, 23 G, 27 G). Test parameters are presented in Table 2.

TABLE 2

| | | Intraluminal |
|---|---|---|
| Needle | Length | Diameter |
| Gauge | (mm) | (mm) |
| 18 G | 38 | 0.84 |
| 23 G | 38 | 0.34 |
| 27 G | 38 | 0.21 |

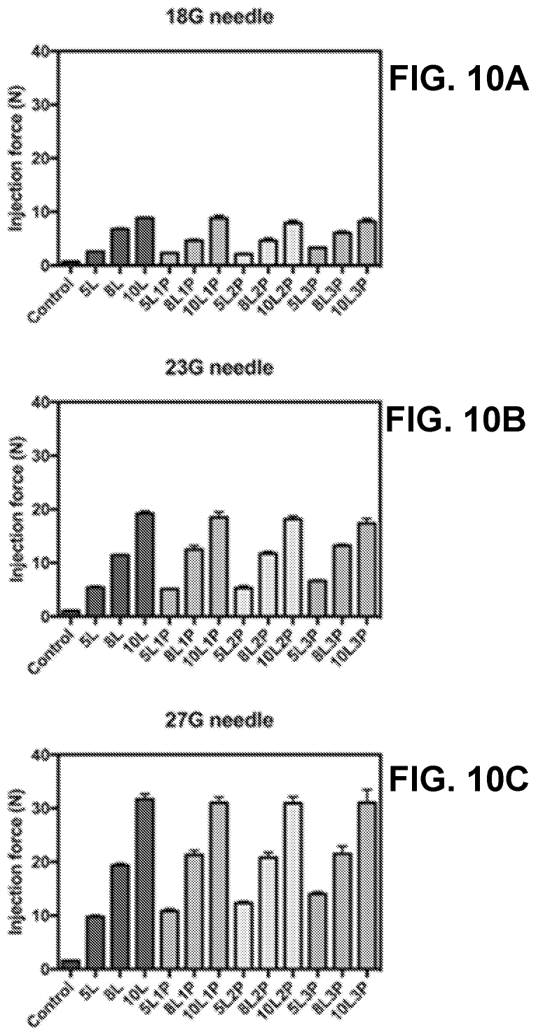

The force needed to extrude the hydrogel was linearly increased until it reached a plateau defining the maximum extrusion force in each formulation, as shown in FIG. 2B. The injection force required to extrude the hydrogel compositions from the syringe was correlated to the needle size and SNP concentration. Increasing SNP concentration (5 wt %, to 8 wt %, and 10 wt %) and decreasing the intraluminal diameter of the needle resulted in an increased extrusion force, as shown in FIG. 2C. The addition of several PEO concentrations (1, 2, and 3 wt %) to SNPs did not significantly increase the required injection force, as shown in FIGS. 10A-10C. All STHBs presented an injection force well below the maximum recommended standard for injectable medical materials (˜80 N) (see e.g., Vo et al, J. Med. Eng. 2016, 2016:5162394).

A spraying system equipped with a nozzle size of 22 G was used to spray the 5L3P, 8L3P, and 10L3P formulations, and average sprayed area and spot size was quantified as previously reported, as shown in FIGS. 2D-2E (see e.g., Sakuma et al, Chemistry Letters 2018, 47(1):68-70). The total area covered after spraying 5L3P was 1.25±0.03 cm², higher viscosity formulations resulted in decreased areas, 0.98±0.09 cm² for 8L3P and 0.45±0.02 cm² for 10L3P respectively, as shown in FIG. 2F. Distribution and average spot size were captured and quantified by micrograph area fraction analysis, as shown in FIG. 2G. Higher viscosity formulations had higher spot areas; 0.013±0.001 mm² for 5L3P, 0.026±0.002 mm² for 8L3P and 0.040±0.003 mm² for 10L3P, as shown in FIG. 2H. Overall, most STHBs were injectable and sprayable, as shown in Table 3.

TABLE 3

| | | |
|---|---|---|
| Composition | Injectable | Sprayable |

| ID | Yes | No | Yes | No |
|---|---|---|---|---|
| 5L | X | | X | |
| 8L | X | | X | |
| 10L | X | | | X |
| 5L1P | X | | X | |
| 8L1P | X | | X | |
| 10L1P | X | | | X |
| 5L2P | X | | X | |
| 8L2P | X | | X | |
| 10L2P | X | | X | |
| 5L3P | X | | X | |
| 8L3P | X | | X | |
| 10L3P | X | | X | |

### Example 5. Spreadability Test

To further study STHBs topical applications, a spreading analysis was performed as previously reported (see e.g., Lardy et al, Drug Development and Industrial Pharmacy 2000, 26(7):715-721). The spreadability of the hydrogels was evaluated 48 hours after their preparation. STHB formulations were incubated for 1 hour at 37° C., and after incubation, the studies were quickly performed at room temperature. STHBs were placed between two horizontal transparent glass plates (Bio-Rad, Hercules, Calif.), and a 125 gram weight was placed on the upper plate. After one minute, the weight was removed, and the spreading diameter measured.

Spreadability was quantified as the total diameter covered by the hydrogel within the plates, and its fluidity and stiffness classified according to a previous published work (see e.g., Lardy et al, Drug Development and Industrial Pharmacy 2000, 26(7):715-721). The spreadability was quantified by placing hydrogels in a device where a standard weight (125 grams) was applied, as shown in FIG. 2I, and classified as fluid gels, semifluid gels, semistiff gels, stiff gels, or very stiff gels based on their spreadability area, as shown in Table 4.

TABLE 4

| | Diameter | Composition |
|---|---|---|
| Classification | (mm) | ID |
| Very Stiff Gel | <40 | 8L, 8L1P, 8L2P, 8L3P, 10L, |
| | | 10L1P, 10L2P, 10L3P, |
| Stiff Gel | 40-47 | 5L, 5L1P, 5L2P, 5L3P, |
| Semistiff Gel | 47-55 | No compositions in this range |
| Semifluid Gel | 55-70 | No compositions in this range |
| Fluid Gel | >70 | No compositions in this range |

The resulting spread diameter of STHB compositions with 5 wt % SNP was in the range of 37.9 to 45.9 mm, 26.9 to 31.4 mm for 8 wt % SNP compositions, and 23.3 to 27.5 mm for 10 wt % SNP compositions, respectively, as shown in FIG. 2J. No significant variation on spreadability was observed when PEO was included in the compositions, and formulations with higher SNP concentration resulted in less spread area; 5 wt % SNP formulations were classified as stiff gels, 8 wt % and 10 wt % SNP formulations were classified as very stiff gels. From these tests, it was concluded that STHBs have sufficient mechanical properties to form spreadable barriers suitable for topical administration.

### Example 6. STHB Stability and Swelling Test

To determine the stability and swelling ratio of STHB formulations, one gram of each formulation was placed in a cell strainer (Corning, Corning, N.Y.) (n=3). Each strainer was submerged in 7 mL of PBS on 6-well plates (Corning, Corning, N.Y.) and incubated at 37° C. Stability and swelling were recorded at 3, 7, 14, and 21 days by quantifying the wet weight and dry weight after lyophilization of STHB compositions. Stability was calculated with the formula: mass loss percentage=(M0−Md)/M0×100%, and swelling ratio (Q) with the formula: Q=(Ms−Md)/Md (see e.g., Tang et al, Journal of Applied Polymer Science 2007, 104(5):2785-2791). M0 represents the original mass of the hydrogel before immersing it into the medium, Ms is the mass of the hydrogel in the swollen state and Md is the mass of the hydrogel in the dry state.

After 21 days, the mass loss of STHB formulations was below 3%, as shown in FIG. 2K), and the maximum swelling ratio (Q) was 15.3 for 5L3P, 13.3 for 8L3P, and 12.7 for 10L3P, respectively, as shown in FIG. 2L. This stable degradation behavior is consistent with previous literature describing a stabilization effect of the suspension due to coverage of the nanoplatelets surface by polymers (see e.g., Zulian et al, Philosophical Magazine 2008, 88(33-35):4213-4221).

### Example 7. Cellular Adherence Assay

Prevention of cell adherence and infiltration to the hydrogel formulations is fundamental for the creation of an effective adhesion barrier. For this purpose, the cell-material interactions between STHBs and the effector cells (fibroblasts) of the formation of postoperative adhesions were investigated.

To determine cellular adherence to the hydrogel compositions, 3T3 fibroblasts were seeded on hydrogel films. 24-well culture plates (Corning Inc., Corning, N.Y.) were coated with 0.2 mL of each hydrogel formulation, and 10×10⁴ cells were seeded. 24 hours after seeding, fibroblasts were washed with PBS to remove unattached cells. Remaining fibroblasts were detached with trypsin (Sigma-Aldrich, Darmstadt, Germany) for posterior quantification. Fibroblast numbers were determined by PrestoBlue Cell Viability Reagent (Thermo Fisher Scientific, Waltham, Mass.) via a microplate reader (BioTek Synergy 2, Winooski, Vt.). Analysis was performed by using BioTek Gen5 software (BioTek Synergy 2, Winooski, Vt.).

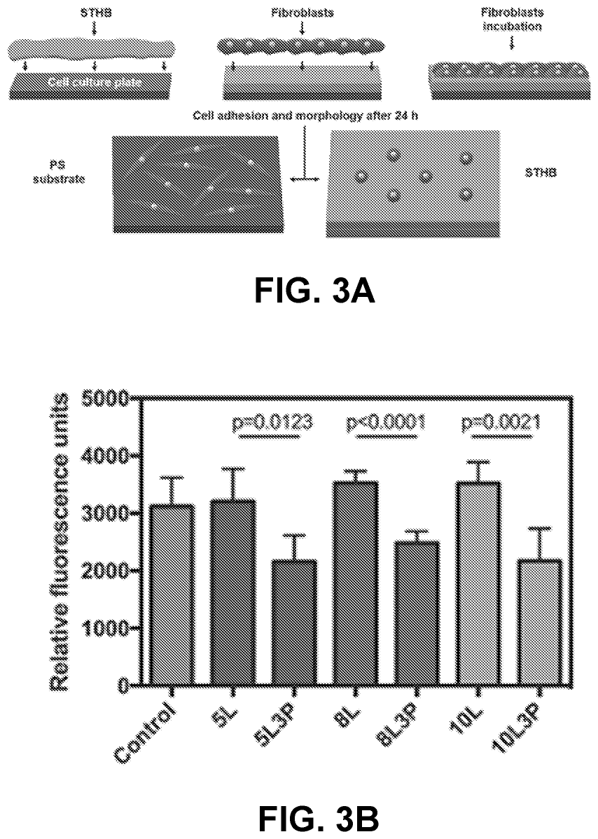

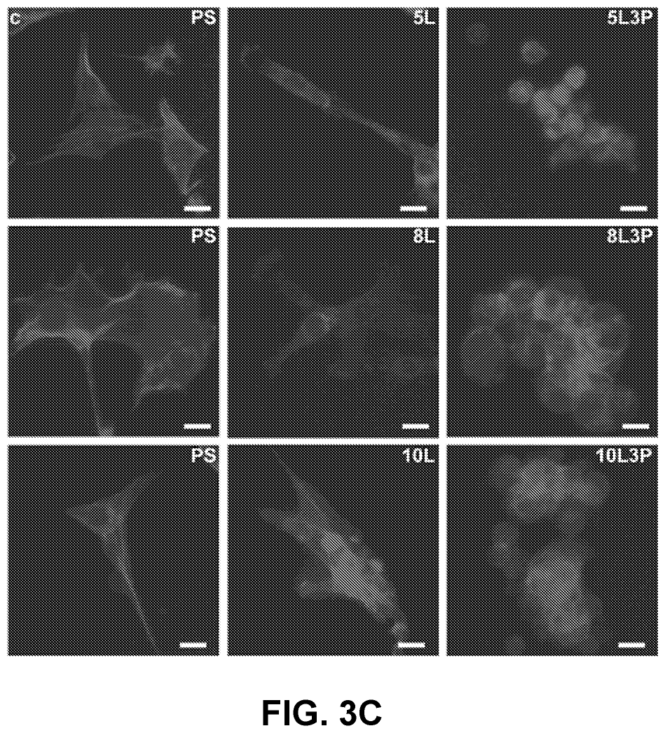

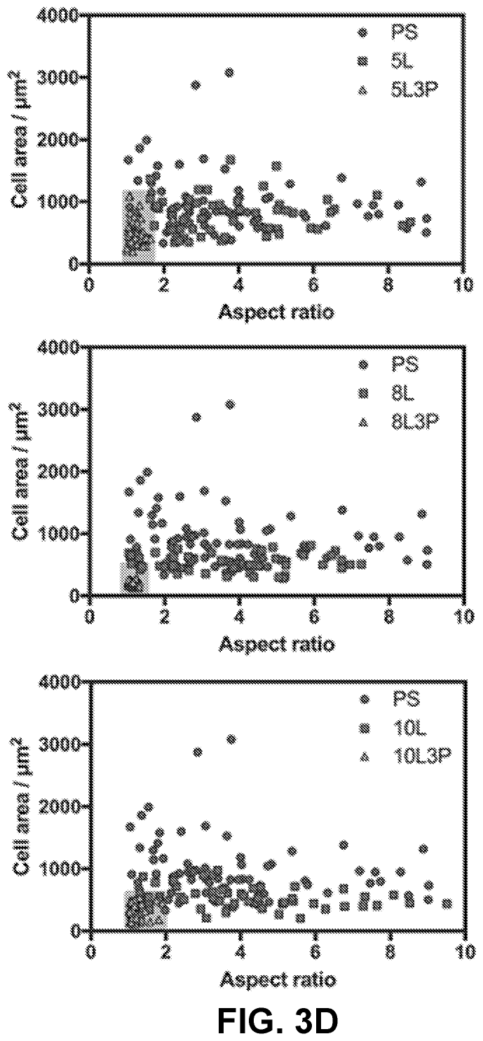

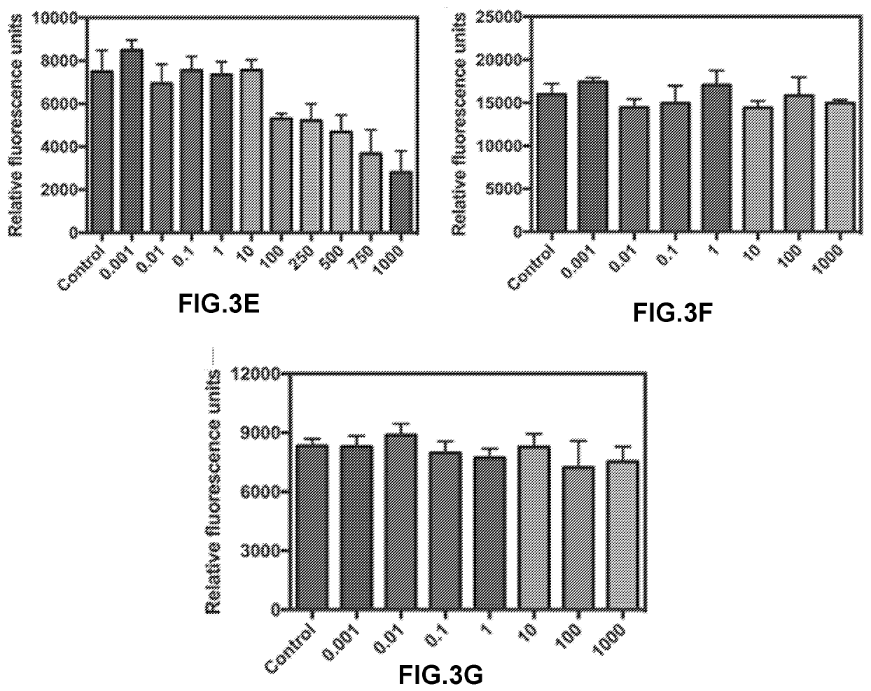

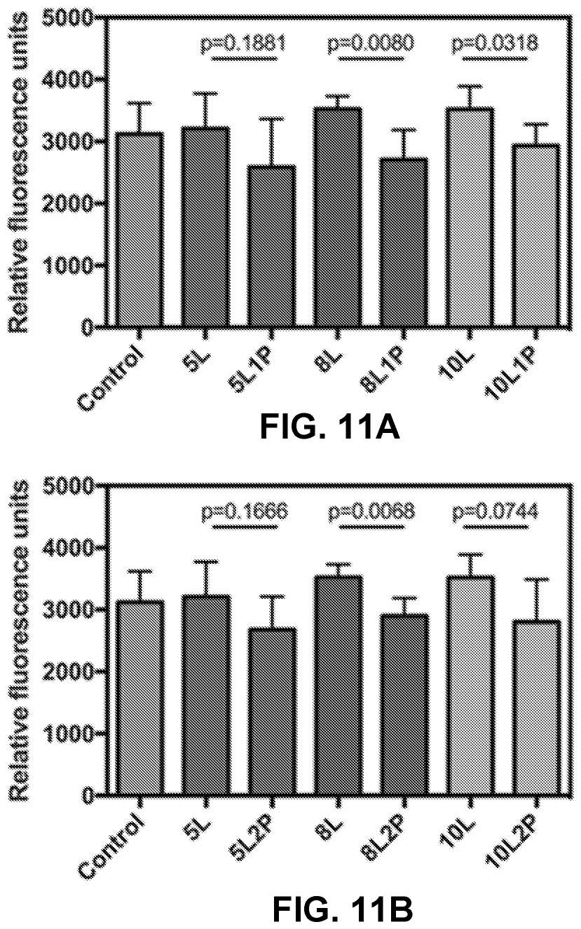

As a control, cells were seeded on polytetrafluoroethylene (PTFE) coated substrates suitable for cell adherence and compared to cells seeded on STHB surfaces, as shown in FIG. 3A. After 24 hours, the emitted relative fluorescence units showed similar cell numbers attached to SNP-only formulations (5L, 8L and 10L) and the control group, however, cell numbers decreased as PEO concentrations increased from 1 wt % to 3 wt %, as shown in FIG. 3B and FIGS. 11A-11B. Formulations with 3 wt % PEO presented 32.8% (5L3P), 29.5% (8L3P), and 38.3% (10L3P) less cells, compared to 5L, 8L and 10L. Based on these results we determined that the most significant and efficacious formulations to prevent cell adherence were the ones containing 3 wt % PEO.

### Example 8. Single Cell Analysis

Cellular morphology was evaluated after seeding 10×10⁴ 3T3 fibroblast on the surface of STHB formulations placed on 6-well culture plates (Corning Inc., Corning, N.Y.). After being incubated for 24 hours, cells were fixed using a 4% paraformaldehyde solution (Sigma-Aldrich, St. Louis, Mo.), followed by F-actin (red) (Thermo Fisher Scientific, Waltham, Mass.) and DAPI (Sigma-Aldrich, St. Louis, Mo.) staining. Cellular fluorescent micrographs at different locations of the material surfaces were obtained using a fluorescence microscope (Zeiss, Oberkochen, Germany), and analyzed by Snap 2058-Zen Pro 2012 software (Zeiss, Oberkochen, Germany). Sixty individual cells per group were randomly selected in each micrograph for analysis. The maximum orthogonal length, width and area of each cell was measured using ImageJ software (National Institutes of Health, Bethesda, Md.), and the aspect ratio was calculated as the longer length divided by the shorter length.

The aspect ratio (based on dimensions, geometry and area) of single cells was quantified to understand cell adherence and expansion (see e.g., Collins et al, Proc. Natl. Acad. Sci. U.S.A. 2017, 114(29):E5835-E5844). Single-cell analysis (aspect ratio and morphology) was performed via F-actin fluorescence labeling. Fluorescent micrographs showed that cells seeded on PTFE substrates (control) and SNP-only formulations (5L, 8L, 10L), had normal cell adherence and pseudopodia expansion, as shown in FIG. 3C. In contrast, when cells were seeded on STHBs with 3 wt % PEO (5L3P, 8L3P, 10L3P), the cellular morphology was spherical, as cells were unable to attach to the hydrogel surface. Previous studies have shown PEO as an efficient polymer to suppress non-specific protein adsorption, and prevent cell adhesion (see e.g., Zhang et al, Biomaterials 1998, 19(10):953-60).

Cells in control and SNP only groups presented a disparate set of aspect ratios on the graphs shown in FIG. 3D; this was expected, as individual attached cells normally differ in shape due to unique pseudopodia expansion when not in confluency (see e.g., Collins et al, Proc. Natl. Acad. Sci. U.S.A. 2017, 114(29):E5835-E5844). Nevertheless, when cells were seeded on 5L3P, 8L3P and 10L3P, most cells had similar aspect ratio indicating spherical morphology and low adhesion.

### Example 9. Cell Viability Test

Cytotoxicity of STHB formulations was evaluated, 10×10³ 3T3 fibroblasts were seeded in 96-well culture plates (Corning Inc., Corning, N.Y.) and incubated for 48 hours with the following ranges of SNPs, PEO and SNPs combined with PEO: 0.001 to 1000 μg/mL. After the incubation period, cell viability was quantified by PrestoBlue Cell Viability Reagent (Thermo Fisher Scientific, Waltham, Mass.) via a microplate reader (BioTek Synergy 2, Winooski, Vt.) and BioTek Gen5 software (BioTek Synergy 2, Winooski, Vt.).

Biocompatibility of STHB compositions was assessed on fibroblasts treated with SNPs, PEO, and a combination of both components (0.001 to 1000 μg/mL). As shown in FIG. 3E, no significant impact on cell viability was observed when SNP concentrations were increased from 0.001 to 100 μg/mL, however, cell viability decreased to 70.8% at 100 μg/mL, and 37.4% at 1000 μg/mL. In contrast, PEO maintained cell viability with minimal toxicity at all ranges, as shown in FIG. 3F. Notably, when a mixture of SNPs and PEO was administered, no cytotoxicity was detected and excellent biocompatibility is achieved, as PEO was capable of mediating SNP-cell interactions and intracellular uptake, FIG. 3G (see e.g., Chan et al, International Journal of Polymer Science 2011, 2011:9).

### Example 10. Peritoneal Adhesion Model

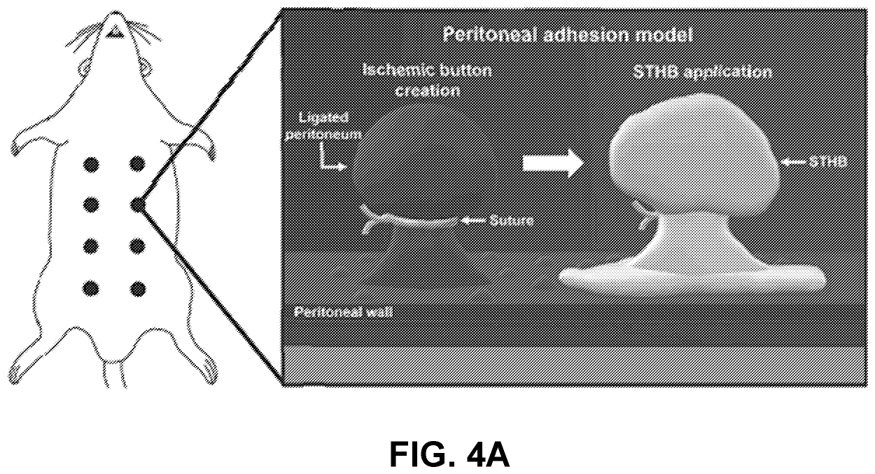

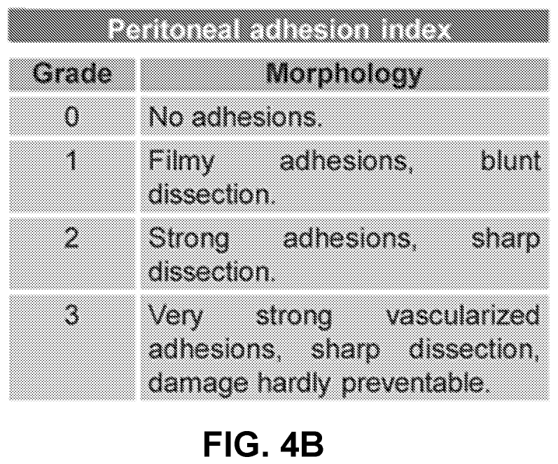

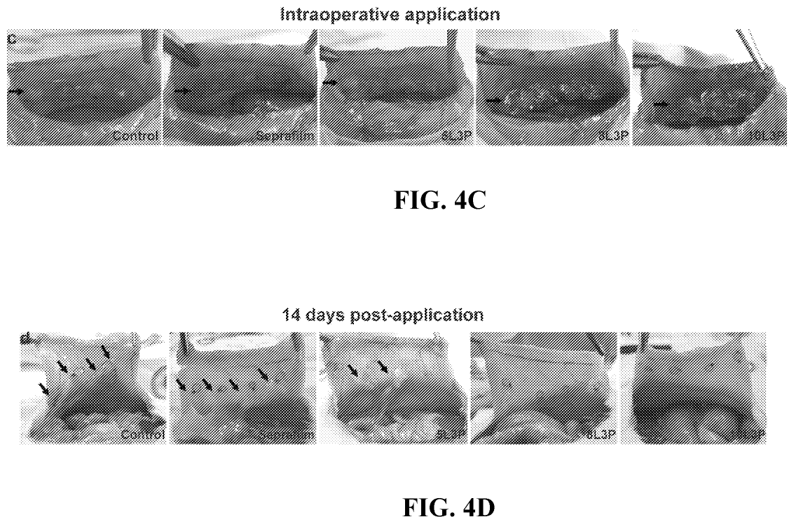

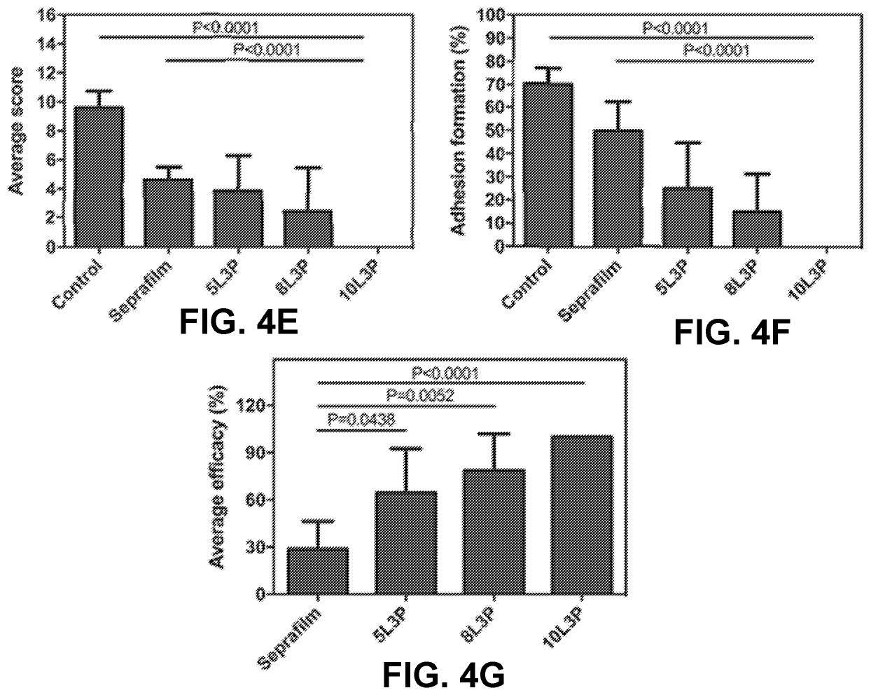

To investigate STHB efficacy in preventing postoperative adhesions, a peritoneal injury rat model (300 g male Wistar rats (Charles River Laboratories, Worcester, Mass.) with 8 ischemic peritoneal buttons was used (FIG. 4A) (see e.g., Whang et al, J. Surg. Res. 2011, 167(2):245-250). Based on the in vitro results, the three most efficient STHB formulations (5L3P, 8L3P and 10L3P) were selected and compared to Seprafilm® (Sanofi, Paris, France) and a control group without any treatment. In total, five groups were tested, each group was composed of 5 animals. After aseptic animal preparation and inhalable isoflurane as anesthesia, a laparotomy was performed using a standard midline incision of 4 cm, and eight peritoneal ischemic buttons were created using a chain distribution in parietal peritoneum (4 per side). Each button was generated by using a Z-stitch technique with 2-0 polypropylene sutures (Ethicon, Somerville, N.J.), and ligating 5 mm of peritoneal tissue. Afterwards, 0.1 mL of STHB or Seprafilm® was placed on the surface of the ischemic buttons. Finally, the peritoneum of the abdominal incision was closed, and the skin layers were sutured separately. Analgesia was administered in the form of carprofen and buprenorphine during the first 48 hours after surgery. Animals were kept alive for 14 days before adhesion formation was assessed.

### Example 11. Determination and Grading of Postoperative Adhesions

The Peritoneal Adhesion Index (PAI) was used as a scoring system to grade the adhesions based on several morphological features such as vascularization, thickness, strength, and damage, as shown in FIG. 4B (see e.g., Coccolini et al, World J. Emerg. Surg. 2013, 8(1):6). Surgically created ischemic buttons and application of Seprafilm®, 5L3P, 8L3P, and 10L3P can be observed in FIG. 4C. During administration, STHBs formed robust coatings that were able to properly adhere and remain in the tissue. After 14 days, animals were sacrificed, and the severity of the adhesions was assessed by the Peritoneal Adhesion Index (PAI).

The severity of adhesions was graded with the following scoring system: 0—no adhesion, 1—filmy adhesion that needs blunt dissection, 2—strong adhesion that needs sharp dissection, 3—very strong vascularized adhesion that needs sharp dissection with damage hardly preventable. Each button (8) was individually graded and the index was calculated based on the sum of the total score of the eight buttons. Percentage of adhesion formation and efficacy of adhesion prevention was calculated based on the number of adhesions formed, each button was considered as 12.5% of the total number of injuries created (8 ischemic buttons).

In the control group, adhesions were found attached to the ischemic button and its surroundings. Seprafilm®, 5L3P, and 8L3P groups had lower adhesion grades, whereas 10L3P group did not develop any observable adhesions, as shown in FIG. 4D.

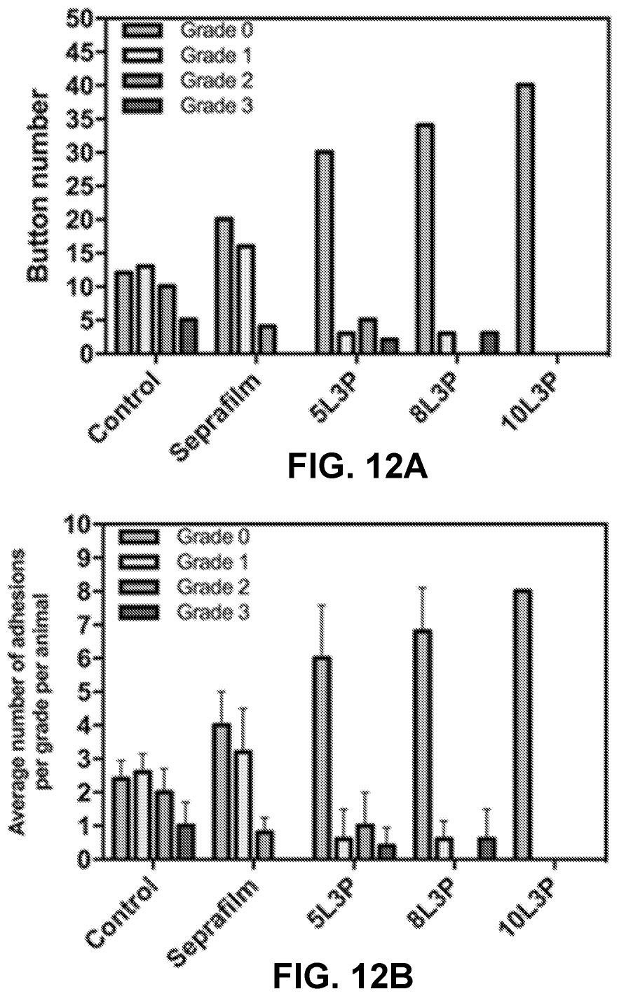

To perform a quantitative analysis of the severity of adhesion formation in all the groups, parameters such as adhesion grade, number, percentage of adhesion formation and efficacy of adhesion prevention were examined. The total average PAI score per group was 9.6±0.5 for control, 4.6±0.4 for Seprafilm®, 6.6±1.9 for 5L3P, 2.4±1.4 for 8L3P, and 0 for 10L3P, as shown in FIG. 4E. In FIGS. 12A-12B, the average numbers of adhesions per grade on each group are detailed.

The average percentage of adhesion formation per group was 70%±3 for control, 50%±5 for Seprafilm®, 38%+8 for 5L3P, 15% 7 for 8L3P, and 0% for 10L3P, as shown in FIG. 4F. Based on these numbers, the average efficacy of Seprafilm® was 28%, 64% for 5L3P, 78% for 8L3P and 100% for 10L3P, as shown in FIG. 4G. Formulations with higher viscosity presented less adhesion formation, with the most viscous composition (10L3P) providing an efficient barrier to inhibit cell infiltration and fibrotic adhesion formation.

### Example 12. Histology and Immunohistology

After 14 days, histopathological examination was performed to evaluate tissue remodeling, inflammatory response and STHB absorption. Peritoneal button tissues were extracted, frozen, and sectioned into 7 μm sagittal and transversal slices using a HM550 Cryostat system (Thermo Fischer Scientific, Waltham, Mass.). Sections were stained with Hematoxylin & Eosin (Sigma-Aldrich, St. Louis, Mo.) and Masson's trichrome (Sigma-Aldrich, St. Louis, Mo.) to assess tissue morphological changes and fibrotic formation. Anti-CD68 (Abcam, Cambridge, Mass.), and anti-CD3 (Abcam, Cambridge, Mass.) primary antibodies with Alexa Fluor-conjugated (Invitrogen, Carlsbad, Calif.) secondary antibodies, were used in conjunction with DAPI (Vector Laboratories, Burlingame, Calif.) to perform immunohistology. Slides were examined (n=5 pictures per section) using an Axio Observer microscope (Zeiss, Oberkochen, Germany).

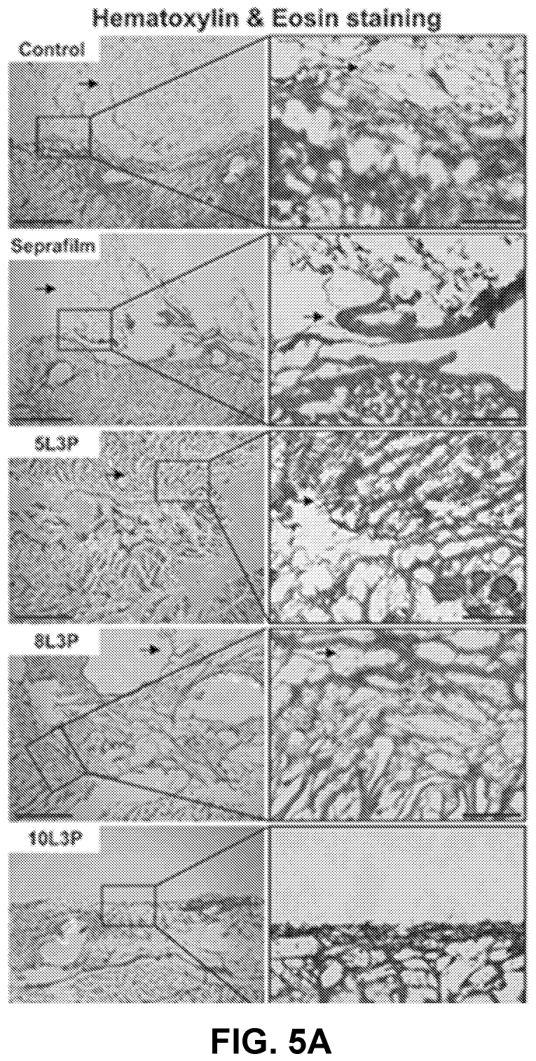

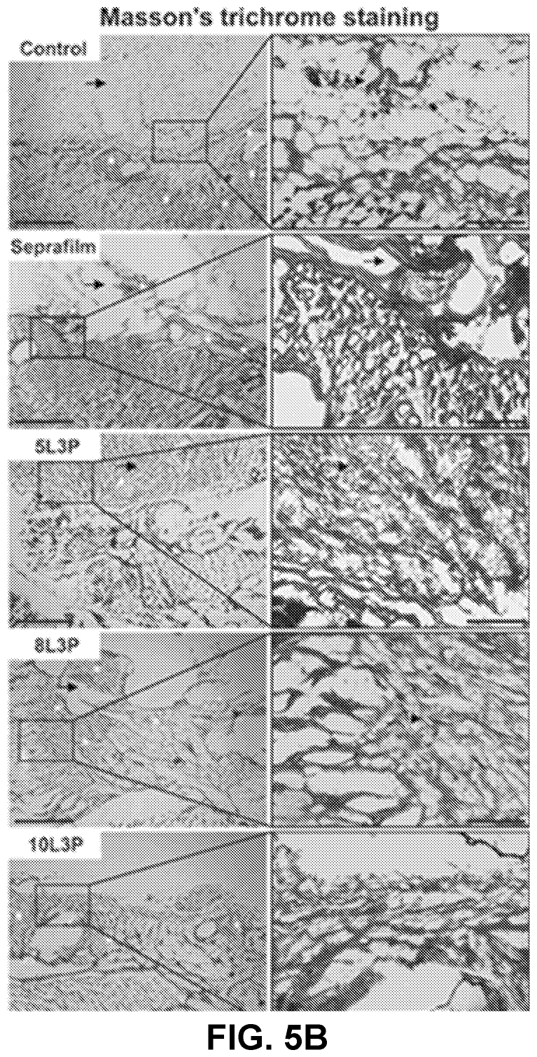

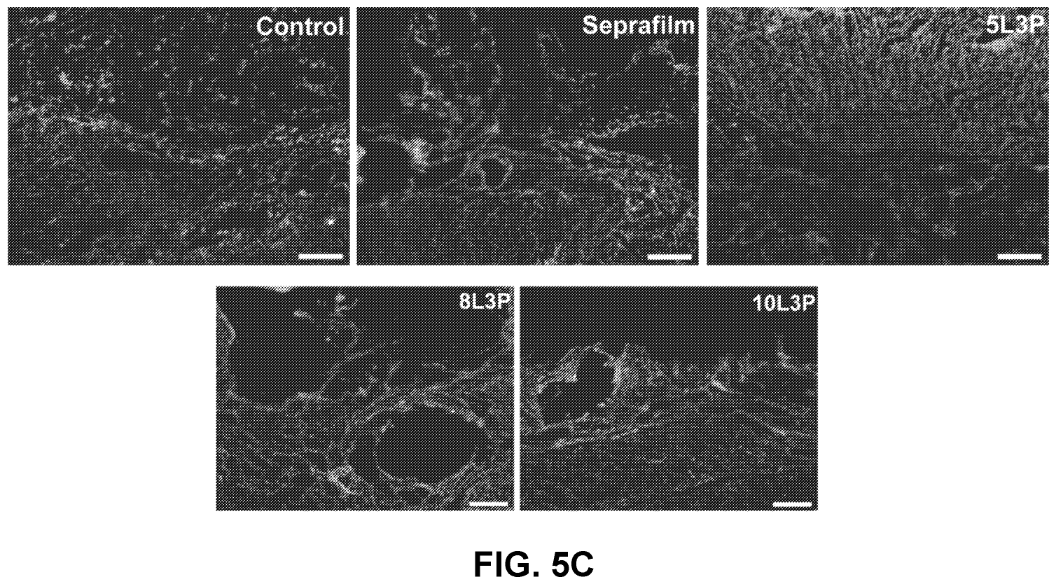

As shown in FIG. 5A, control group, a grade 3 adhesion with fibrotic bands adhered to the mesothelial lining where the ischemic button was created is presented. In the Seprafilm® group, a formation of a thin fibrotic band connected to the peritoneal wall is shown, confirming the low-grade adhesions that were observed during the PAI analysis. A grade 3 adhesion composed of a thick fibrotic band attached to the mesothelium was observed on 5L3P. 8L3P micrograph showed a grade 1 adhesion with filmy morphology and undefined interface with the peritoneal lining. Group 10L3P did not show any adhesion formation, the mesothelial lining was maintained without fibrotic band formation.

The abdominal cavity of the animal was also examined and no hydrogel or Seprafilm® barrier was found. H&E examination confirmed its degradation in a period of 2 weeks. Rapid resorption is an important requirement for an adhesion barrier material, as lengthy residence time in the peritoneal cavity can retard re-mesothelialization of the peritoneal lining.

Masson's trichrome staining was also performed to determine the presence of fibrotic bands in the peritoneal wall, as shown in FIG. 5B. Control group micrograph shows several blue stained collagen fibrotic areas. Seprafilm® presented collagen deposition mainly in the adhesion and peritoneal interface areas. The bands found on 5L3P indicated a thick collagen deposition adhered to the peritoneal lining. As compared to previous groups, 8L3P micrographs showed less dense and non-specifically distributed collagen areas consistent with local tissue remodeling. The 10L3P micrograph showed a less dense collagen deposition and a restored mesothelial lining at 2 weeks after the injury.

Local inflammatory response was analyzed by CD3 and CD68 immunostaining, as shown in FIG. 5C. The control group exhibited minimal leukocyte and macrophage infiltration. Micrographs from Seprafilm® and STHB groups showed a similar pattern with negligible local inflammatory response at the surgical injury site.

These observations confirmed that STHB formulations were superior in preventing adhesions compared to a commercially available barrier, and biocompatible as no abnormal local immunological response was observed.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition, comprising:
about 3% to about 15% by weight laponite; and
about 0.5% to about 10% by weight poly(ethylene oxide).

2. The composition of claim 1, wherein the composition comprises about 3% to about 10% laponite.

3. The composition of claim 1, wherein the composition comprises about 5% to about 10% laponite.

4. The composition of claim 1, wherein the composition comprises about 8% to about 10% laponite.

5. The composition of any one of claims 1 to 4, wherein the laponite has an average diameter of from about 5 nm to about 60 nm.

6. The composition of any one of claims 1 to 4, wherein the laponite has an average diameter of from about 10 nm to about 40 nm.

7. The composition of any one of claims 1 to 4, wherein the laponite has an average diameter of from about 20 nm to about 30 nm.

8. The composition of any one of claims 1 to 7, wherein the laponite has an average thickness of from about 0.5 nm to about 2 nm.

9. The composition of any one of claims 1 to 7, wherein the laponite has an average thickness of about 1 nm.

10. The composition of any one of claims 1 to 9, wherein the composition comprises about 1% to about 3% by weight poly(ethylene oxide).

11. The composition of any one of claims 1 to 10, wherein the composition comprises about 3% to about 5% by weight poly(ethylene oxide).

12. The composition of any one of claims 1 to 11, wherein the poly(ethylene oxide) has an average molecular weight of about 18,000 to about 22,000 g/mol.

13. The composition of claim 1, wherein the composition comprises:
about 8% to about 10% by weight laponite and about 1% to about 5% by weight poly(ethylene oxide); or
about 5% to about 10% by weight laponite and about 1% to about 3% by weight poly(ethylene oxide); or
about 8% to about 10% by weight laponite and about 1% to about 3% by weight poly(ethylene oxide).

14. The composition of claim 1, wherein the composition comprises:
about 5% by weight laponite and about 1% by weight poly(ethylene oxide); or
about 8% by weight laponite and about 1% by weight poly(ethylene oxide); or
about 10% by weight laponite and about 1% by weight poly(ethylene oxide); or
about 5% by weight laponite and about 2% by weight poly(ethylene oxide); or
about 8% by weight laponite and about 2% by weight poly(ethylene oxide); or
about 10% by weight laponite and about 2% by weight poly(ethylene oxide); or
about 5% by weight laponite and about 3% by weight poly(ethylene oxide); or
about 8% by weight laponite and about 3% by weight poly(ethylene oxide); or
about 10% by weight laponite and about 3% by weight poly(ethylene oxide).

15. The composition of any one of claims 1 to 14, wherein the composition further comprises water.

16. The composition of claim 15, wherein the composition comprises about 85% to about 94% by weight water.

17. The composition of claim 16, wherein the composition comprises:
about 8% to about 10% by weight laponite, about 1% to about 5% by weight poly(ethylene oxide), and about 85% to about 91% by weight water; or
about 5% to about 10% by weight laponite, about 1% to about 3% by weight poly(ethylene oxide), and about 87% to about 94% by weight water; or
about 8% to about 10% by weight laponite, about 1% to about 3% by weight poly(ethylene oxide), and about 87% to about 91% by weight water.

18. The composition of any one of claims 15 to 18, wherein the composition consists of laponite, poly(ethylene oxide), and water.

19. The composition of any one of claims 15 to 18, wherein the water is deionized water.

20. The composition of any one of claims 15 to 19, wherein the composition is a gel.

21. The composition of any one of claims 15 to 20, wherein the composition is a hydrogel.

22. The composition of any one of claims 1 to 21, wherein the laponite consists of about 66% SiO₂, about 30% MgO, about 3% Na₂O, and about 1% LiO₂.

23. A composition comprising about 5% to about 10% by weight laponite, about 1% to about 5% by weight poly(ethylene oxide), and about 85% to about 94% by weight water, wherein the composition is prepared according to a process comprising:
(a) combining the laponite and water to form a first mixture;
(b) adding the poly(ethylene oxide) to the first mixture to form the composition.

24. The composition of any one of claims 1 to 23, wherein the yield stress of the composition is from about 100 Pa to about 2000 Pa.

25. A kit comprising a composition of any one of claims 1 to 24.

26. The kit of claim 25, wherein the kit further comprises one or more sterile syringes.

27. The kit of claim 26, wherein the composition is preloaded into the one or more sterile syringes.

28. The kit of any one of claims 25 to 27, wherein the kit further comprises one or more sterile bandages.

29. The kit of claim 28, wherein the composition is preloaded onto a surface of the one or more sterile bandages.

30. The kit of any one of claims 25 to 29, wherein the kit further comprises one or more sterile surgical staples.

31. The kit of claim 30, wherein the composition is preloaded onto a surface of the one or more sterile surgical staples.

32. The kit of any one of claims 25 to 31, wherein the kit further comprises one or more sterile surgical sutures.

33. The kit of claim 32, wherein the composition is preloaded onto a surface of the one or more sterile surgical sutures.

34. A sterile syringe comprising a composition of any one of claims 1 to 24.

35. A sterile bandage, comprising a composition of any one of claims 1 to 24.

36. A sterile surgical staple, comprising a composition of any one of claims 1 to 24.

37. A sterile surgical suture, comprising a composition of any one of claims 1 to 29.

38. A coating, comprising a pharmaceutically acceptable amount of a composition of any one of claims 1 to 24.

39. The coating of claim 38, wherein the coating is preloaded into a sterile syringe.

40. The coating of claim 38, wherein the coating is preloaded onto a surface of a sterile bandage.

41. The coating of claim 38, wherein the coating is preloaded onto a surface of a sterile surgical staple.

42. The coating of claim 38, wherein the coating is preloaded onto a surface of a sterile surgical suture.

43. A method of reducing or preventing tissue adhesion in a subject, comprising administering to the subject a composition of any one of claims 1 to 24.

44. The method of claim 43, wherein the tissue adhesion is associated with a surgical procedure, an injury, an anatomical defect, a cosmetic defect, or any combination thereof.

45. The method of claim 43 or 44, wherein the composition forms a barrier between two or more tissues in the subject, thereby reducing or preventing the tissue adhesion.

46. A method of reducing or preventing fibrotic adhesion formation in a subject, comprising administering to the subject a composition of any one of claims 1 to 24.

47. The method of any one of claims 43 to 46, wherein the composition is administered by injection.

48. The method of any one of claims 43 to 47, wherein the composition is administered as a sprayable composition.

49. The method of any one of claims 43 to 48, wherein the composition is administered using a syringe.

50. The method of any one of claims 43 to 49, wherein the method is associated with one or more of surgical, cosmetic, orthopedic, ophthalmic, and dermal applications.

51. The method of any one of claims 43 to 50, wherein the composition is administered during a surgical procedure.

52. The method of claim 51, wherein the surgical procedure is associated with one or more of abdominal, thoracic, pelvic, vascular, cardiovascular, neurological, and dermal surgical procedures.

53. The method of any one of claims 43 to 52, wherein the method comprises local administration at a tissue associated with a surgical procedure, an injury, an anatomical defect, a cosmetic defect, or any combination thereof.

54. The method of any one of claims 51 to 53, wherein the surgical procedure is selected from the group consisting of a laparoscopic surgical procedure and an arthroscopic guided surgical procedure.

55. The method of any one of claims 51 to 54, wherein the surgical procedure is selected from the group consisting of laparoscopic myomectomy, ileal pouch anal-anastomosis, cesarean section, nerve repair, hernia repair, cardiac surgery, spinal surgery.

56. The method of any one of claims 43 to 55, wherein the composition is administered using a catheter.

57. The method of any one of claims 43 to 56, wherein the method comprises reducing or preventing the closure of permanent surgical central access lines, peripheral access lines, catheter lines, or drain lines, in the subject.

58. The method of any one of claims 43 to 57, wherein the composition is locally administered at the site of a surgical suture or surgical staple in the subject.

59. The method of any one of claims 46 to 58, wherein the composition forms a barrier between two or more tissues in the subject, thereby reducing or preventing the fibrotic adhesion formation.
